SEQUENCE LISTING (34 sequences)

SEQ ID NO: 1 - DNA, 36 bp, Artificial Sequence
  Description of Artificial Sequence Synthetic oligonucleotide
aaggtgacactatagaatgccgtgggaccatacagg

SEQ ID NO: 2 - DNA, 43 bp, Artificial Sequence
  Description of Artificial Sequence Synthetic oligonucleotide
gtacgactcactatagggaaagtgatctgtagcttggaatctc

SEQ ID NO: 3 - DNA, 41 bp, Artificial Sequence
  Description of Artificial Sequence Synthetic oligonucleotide
aggtgacactatagaataagggtaactcacagcaaactcaa

SEQ ID NO: 4 - DNA, 37 bp, Artificial Sequence
  Description of Artificial Sequence Synthetic oligonucleotide
gtacgactcactatagggagagcagcctgtccatccc

SEQ ID NO: 5 - DNA, 38 bp, Artificial Sequence
  Description of Artificial Sequence Synthetic oligonucleotide
aggtgacactatagaataaaactactggacgggcttga

SEQ ID NO: 6 - DNA, 38 bp, Artificial Sequence
  Description of Artificial Sequence Synthetic oligonucleotide
gtacgactcactatagggatgagatgcccaaatgttgc

SEQ ID NO: 7 - DNA, 36 bp, Artificial Sequence
  Description of Artificial Sequence Synthetic oligonucleotide
aggtgacactatagaatagtgagttcgttggatggc

SEQ ID NO: 8 - DNA, 44 bp, Artificial Sequence
  Description of Artificial Sequence Synthetic oligonucleotide
gtacgactcactatagggatatgttttgtataagagttcatttg

SEQ ID NO: 9 - DNA, 40 bp, Artificial Sequence
  Description of Artificial Sequence Synthetic oligonucleotide
aggtgacactatagaatacagtggcttccattagaagcat

SEQ ID NO: 10 - DNA, 37 bp, Artificial Sequence
  Description of Artificial Sequence Synthetic oligonucleotide
gtacgactcactatagggaggtcacatcctctcacta

SEQ ID NO: 11 - DNA, 36 bp, Artificial Sequence
  Description of Artificial Sequence Synthetic oligonucleotide
aggtgacactatagaatactcaggtgcgaaagcgtg

SEQ ID NO: 12 - DNA, 40 bp, Artificial Sequence
  Description of Artificial Sequence Synthetic oligonucleotide
gtacgactcactatagggacgttgcatcgaattaaaccac

SEQ ID NO: 13 - DNA, 41 bp, Artificial Sequence
  Description of Artificial Sequence Synthetic oligonucleotide
aggtgacactatagaatagcgtcatttacaaggagaacatc

SEQ ID NO: 14 - DNA, 36 bp, Artificial Sequence
  Description of Artificial Sequence Synthetic oligonucleotide
gtacgactcactatagggaatctcgcccatgcccac

SEQ ID NO: 15 - DNA, 44 bp, Artificial Sequence
  Description of Artificial Sequence Synthetic oligonucleotide
aggtgacactatagaatatggtttgagaacagagaaataataga

SEQ ID NO: 16 - DNA, 36 bp, Artificial Sequence
  Description of Artificial Sequence Synthetic oligonucleotide
gtacgactcactatagggagcttgttgccgggggtc

SEQ ID NO: 17 - DNA, 127 bp, Foot and mouth disease virus
gccgtgggaccatacaggagaagttgatctccgtggcaggactcgccgtccactctggacctgacgagtaccggcgtctcttcgagcccttccagggcctctttgagattccaagctacagatcact

SEQ ID NO: 18 - DNA, 99 bp, Bluetongue virus
agggtaactcacagcaaactcaagcaggcgtgactgttagcgttggtggagtagatatgcgggcggggcgtatcatagcgtgggatggacaggctgctc

SEQ ID NO: 19 - DNA, 241 bp, Vesicular stomatitis virus
aaactactggacgggcttgaaaatcagtgcaaagtggcgtcaaccagatttgagagtctagtcgaggatggtctcgacttctttgacatatgggagaatgatccaaatttcaccaagatagttgctgcagtggatatgttcttccacatgttcaaaaagcatgaacgtgctccaatcagatacggaaccatagtctcaagattcaaggactgtgcagcacttgcaacatttgggcatctca

SEQ ID NO: 20 - DNA, 271 bp, Bovine viral diarrhea virus
gtgagttcgttggatggctgaagccctgagtacagggtagtcgtcagtggttcgacgctttgtgcgacaagcctcgagatgccacgtggacgagggcatgcccacagcacatcttaacctgagcgggggtcgttcaggtgaaaacggtttaaccaaccgctacgaatacagcctgatagggtgctgcagaggcccactgtatcgctactaaaaatctctgctgtacatggcatatggagttgatcacaaatgaactcttatacaaaacata

SEQ ID NO: 21 - DNA, 174 bp, Bovine rotavirus
cagtggcttccattagaagcatgcttgtcaaatgaggaccaagctaaccacttggtatccgactttggtgagtatgtagctacgtcaagctgtttgaactctgtaagtaaggatgcgtctacgtattcgctacacagagtaatcactcagatggcgtagtgagaggatgtgacc

SEQ ID NO: 22 - DNA, 307 bp, Peste des petits ruminant virus
tggtttgagaacagagaaataatagacatagaggtgcaagatgcagaagagttcaatatgttgctagcctccatcttagcacaagtttggatcctcctggccaaggcggttacggcaccggatacggcagctgactcagaactgagaaggtgggttaaatacacacaacaaaggagagtgattggggaatttcgccttgacaaagggtggctggacgcagtccgcaacaggattgcagaagatctatcacttcggcggttcatggtatctctcatacttgacatcaaaaggacccccggcaacaagc

SEQ ID NO: 23 - DNA, 216 bp, Enterotoxigenic E.coli
ctcaggtgcgaaagcgtggggagcaaacaggattagataccctggtagtccacgccgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacg

SEQ ID NO: 24 - DNA, 151 bp, Infectious bovine rhinotracheitis virus
gcgtcatttacaaggagaacatcgcgccgtacacgttcaaggcctacatttactacaaaaacgtgctcgtgaccgggaaaagcccggggggcacgtacgcggccattacaaaccagtacacggaccgcatgcccgtgggcatgggcgagat

SEQ ID NO: 25 - DNA, 18 bp, Artificial Sequence
  Description of Artificial Sequence Synthetic oligonucleotide
aggtgacactatagaata

SEQ ID NO: 26 - DNA, 19 bp, Artificial Sequence
  Description of Artificial Sequence Synthetic oligonucleotide
gtacgactcactataggga

SEQ ID NO: 27 - DNA, 165 bp, Foot and mouth disease virus
aaggtgacactatagaatgccgtgggaccatacaggagaagttgatctccgtggcaggactcgccgtccactctggacctgacgagtaccggcgtctcttcgagcccttccagggcctctttgagattccaagctacagatcactttccctatagtgagtcgtac

SEQ ID NO: 28 - DNA, 136 bp, Bluetongue virus
aggtgacactatagaataagggtaactcacagcaaactcaagcaggcgtgactgttagcgttggtggagtagatatgcgggcggggcgtatcatagcgtgggatggacaggctgctctccctatagtgagtcgtac

SEQ ID NO: 29 - DNA, 278 bp, Vesicular stomatitis virus
aggtgacactatagaataaaactactggacgggcttgaaaatcagtgcaaagtggcgtcaaccagatttgagagtctagtcgaggatggtctcgacttctttgacatatgggagaatgatccaaatttcaccaagatagttgctgcagtggatatgttcttccacatgttcaaaaagcatgaacgtgctccaatcagatacggaaccatagtctcaagattcaaggactgtgcagcacttgcaacatttgggcatctcatccctatagtgagtcgtac

SEQ ID NO: 30 - DNA, 308 bp, Bovine viral diarrhea virus
aggtgacactatagaatagtgagttcgttggatggctgaagccctgagtacagggtagtcgtcagtggttcgacgctttgtgcgacaagcctcgagatgccacgtggacgagggcatgcccacagcacatcttaacctgagcgggggtcgttcaggtgaaaacggtttaaccaaccgctacgaatacagcctgatagggtgctgcagaggcccactgtatcgctactaaaaatctctgctgtacatggcatatggagttgatcacaaatgaactcttatacaaaacatatccctatagtgagtcgtac

SEQ ID NO: 31 - DNA, 211 bp, Bovine rotavirus
aggtgacactatagaatacagtggcttccattagaagcatgcttgtcaaatgaggaccaagctaaccacttggtatccgactttggtgagtatgtagctacgtcaagctgtttgaactctgtaagtaaggatgcgtctacgtattcgctacacagagtaatcactcagatggcgtagtgagaggatgtgacctccctatagtgagtcgtac

SEQ ID NO: 32 - DNA, 344 bp, Peste des petits ruminant virus
aggtgacactatagaatatggtttgagaacagagaaataatagacatagaggtgcaagatgcagaagagttcaatatgttgctagcctccatcttagcacaagtttggatcctcctggccaaggcggttacggcaccggatacggcagctgactcagaactgagaaggtgggttaaatacacacaacaaaggagagtgattggggaatttcgccttgacaaagggtggctggacgcagtccgcaacaggattgcagaagatctatcacttcggcggttcatggtatctctcatacttgacatcaaaaggacccccggcaacaagctccctatagtgagtcgtac

SEQ ID NO: 33 - DNA, 253 bp, Enterotoxigenic E.coli
aggtgacactatagaatactcaggtgcgaaagcgtggggagcaaacaggattagataccctggtagtccacgccgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgtccctatagtgagtcgtac

SEQ ID NO: 34 - DNA, 188 bp, Infectious bovine rhinotracheitis virus
aggtgacactatagaatagcgtcatttacaaggagaacatcgcgccgtacacgttcaaggcctacatttactacaaaaacgtgctcgtgaccgggaaaagcccggggggcacgtacgcggccattacaaaccagtacacggaccgcatgcccgtgggcatgggcgagattccctatagtgagtcgtac

(19) United States
(12) Patent Application Publication
(10) Pub. No.: US 2018/0291473 A1
(43) Pub. Date: Oct. 11, 2018

(54) A primer combination and GeXP detection method for simultaneously identifying eight kinds of bovine pathogens

(71) Applicant: GUANGXI VETERINARY RESEARCH INSTITUTE, Nanning (CN)

(72) Inventors: Zhixun XIE, Nanning (CN); Qing FAN, Nanning (CN); Zhiqin XIE, Nanning (CN); Xianwen DENG, Nanning (CN); Liji XIE, Nanning (CN); Li HUANG, Nanning (CN); Sisi LUO, Nanning (CN); Jiaoling HUANG, Nanning (CN); Yanfang ZHANG, Nanning (CN); Tingting ZENG, Nanning (CN); Sheng WANG, Nanning (CN); Jiabo LIU, Nanning (CN); Yaoshan PANG, Nanning (CN)

(73) Assignee: GUANGXI VETERINARY RESEARCH INSTITUTE, Nanning (CN)

(21) Appl. No.: 15/567,612

(22) PCT Filed: Apr. 19, 2017
(86) PCT No.: PCT/CN2017/081056

§ 371 Date: Oct. 19, 2017

(30) Foreign Application Priority Data
Jul. 19, 2016 (CN) 2016105694234

(51) Int. Cl. C12Q 1/70; C12Q 1/689

(52) U.S. Cl. CPC C12Q 1/701; C12Q 2600/16; C12Q 1/689

(57) ABSTRACT

The present disclosure discloses a primer combination and GeXP detection method for simultaneously identifying eight kinds of bovine pathogens. The primer combination of the present disclosure consists of primer pair I, primer pair II, primer pair III, primer pair IV, primer pair, primer pair VI, primer pair VII and primer pair VIII. The present disclosure also discloses a GeXP detection method that can simultaneously identify bovine infectious diseases of foot-and-mouth disease virus, bluetongue virus, vesicular stomatitis virus, bovine viral diarrhea virus, bovine rotavirus, enterotoxigenic E. coli, infectious bovine rhinotracheitis virus and peste des petits ruminants virus. The GeXP detection method established can simultaneously identify the eight pathogens of bovine infectious diseases. The method has the characteristics of high throughput, high specificity and sensitivity, and can be used for the bovine epidemiological monitoring and the differential diagnosis of sudden epidemic situation, and guarantees the healthy development of cattle industry.

### CROSS REFERENCE TO RELATED APPLICATIONS

This application is the national phase entry of International Application No. PCT/CN2017/081056, filed on Apr. 19, 2017, which is based upon and claims priority to Chinese patent application No. CN201610569423.4, filed on Jul. 19, 2016, the entire contents of which are hereby incorporated by reference in its entirety.

### TECHNICAL FIELD

The present disclosure relates to a primer combination and a GeXP detection method for simultaneously identifying eight kinds of bovine pathogens.

### BACKGROUND

At present, the number of existing cattle stocks in our country is 138 million, and the yield of beef reaches 6.759 million tons, and our country is the world's fourth largest producer of beef. In recent years, the cattle industry in Guangxi has also fastly developed, with the country's fifth cattle stocks, of which the number of buffalo stocks reaches 4.5 million, accounting for ⅕ of the total number of the national buffalo, ranking first in the country, second in the world. With the development of cattle industry, the incidence of cattle infectious diseases is also increasing year by year, which has become a major factor of restricting the development of cattle industry, the specific performances include: the old disease is still popular, and the pathogen appears a new serotype or mutant strains, the new diseases appear in succession, which bring difficulties to the prevention and control of cattle diseases. A large number of cattle died each year due to the diseases, which causes serious economic losses. According to statistics in 2015, the entire mortality of the annual domestic beef cattle due to infectious disease reaches as high as 5%, resulting in direct economic losses of up to 90-150 billion yuan, the cattle infectious diseases seriously affect China's meat and its products entering, into the international market. Foot and Mouth Disease Virus (FMDV), Bluetongue Virus (BTV), Vesicular Stomatitis Virus (VSV), Bovine Viral Diarrheal Virus (BVDV), Bovine Rotavirus (BRV), Enterotoxigenic E. coli (ETEC), Infectious Bovine Rhinotracheitis Virus (IBRV) and Peste des Petits Ruminants (PPRV) are pathogens of eight major infectious diseases that seriously threaten the cattle industry, and the existence of these pathogens endangers the development of the cattle industry all the time. Bovine foot and mouth disease caused by FMDV, bovine vesicular stomatitis caused by VSV as well as the bovine bluetongue caused by BTV are highly acute infectious disease of cattle, which are generally epidemic outbreak, clinical lesions appear in the mouth, hoof and breast, the symptoms are very similar and are difficult to be distinguished, the mortality are high, which are listed as the infectious diseases of Class A by the World Organization for Animal Health (OIE). BVDV, BRV and ETEC are also the major pathogens causing bovine diarrhea, and BVDV exists in the form of a symptom of persistent infections, and a considerable portion of cattle in the herd are carriers of these pathogens and often break out associated with other diseases, the symptoms of sick cattle are acute watery diarrhea and rapid weight loss. IBRV is an immunosuppressive disease virus, after infection, the organism can also be secondarily infected by bacteria, according to the survey, not only different varieties of cattle infected are reported, but also infected areas spread over provinces and cities nationwide, the infection rate is high. The peste des petits ruminants disease caused by PPRV is also a new disease emerging in recent years, which has been listed as an animal disease by OIE that must be reported, and is classified as the animal diseases of Class A in our country. These diseases are the huge potential trouble of the cattle industry, once outbreak, it will cause huge economic losses, so the rapid development of cattle infectious disease research is imperative.

Rapid and accurate detection of infectious diseases is a prerequisite and basis for effective prevention and control. The main methods used to identify and diagnose these bovine infectious diseases are pathogen isolation and serological tests, however, these methods are often limited by clinical freshness, contamination or serum titers, resulting in erroneous results time-consuming and labour intensive, and have some limitations in practical application. With the progress of molecular biology, molecular biology diagnostic method based on PCR technology has been widely used in the detection and diagnosis of infectious diseases, including PCR, fluorescence PCR and LAMP etc., but these methods can only detect a single or 2 to 4 kinds of pathogens, and cannot achieve the true sense of implement of high-throughput detection for a variety of pathogens at the same time. GeXP multi-gene expression analysis system is a new type of high-throughput gene detection technology, the multiplex PCR and capillary electrophoresis technology could be effectively combined, the universal primers with fluorescent labeling and specific chimeric primers (i.e., gene-specific primer 5′-linked universal primers) are combined to trigger the amplification of the multiplex PCR system, which can simultaneously detect and analyze up to 30 target genes, and achieve the purpose of true sense of high-throughput detection and identification of multiple pathogens.

### SUMMARY

The object of the present disclosure is to provide a primer combination and GeXP detection method for simultaneously identifying eight kinds of bovine pathogens.

The present disclosure provides a primer combination consisting of primer pair I, primer pair II, primer pair III, primer pair IV, primer pair V, primer pair VI, primer pair VII and primer pair VIII;

The primer pair I is composed of the primer FMDV-F and the primer FMDV-R;

The primer FMDV-F is the following (a1) or (a2) or (a3):

(a1) sequence of the single-stranded DNA molecule shown in SEQ ID NO: 1;

(a2) sequence of the single-stranded DNA molecule shown in SEQ ID NO: 1 from the 5′ end of the 19th to 36th nucleotides;

(a3) a DNA molecule having the same function as (a1) or (a2) by substituting and/or deleting and/or adding one or more nucleotides;

The primer FMDV-R is the following (a4) or (a5) or (a6):

(a4) sequence of the single stranded DNA molecule shown in SEQ ID NO: 2;

(a5) sequence of the single stranded DNA molecule shown in SEQ ID NO: 2 from the 5′ end of the 20th to 43th nucleotides;

(a6) a DNA molecule having the same function as (a4) or (a5) by substituting and/or deleting and/or adding one or more nucleotides;

The primer pair II is composed of the primer BTV-F and the primer BTV-R;

The primer BTV-F is the following (a7) or (a8) or (a9):

(a7) sequence of the single stranded DNA molecule shown in SEQ ID NO: 3;

(a8) sequence of the single stranded DNA molecule shown in SEQ ID NO: 3 from the 5′ end of the 19th to 41th nucleotides;

(a9) a DNA molecule having the same function as (a7) or (a8) by substituting and/or deleting and/or adding one or more nucleotides;

The primer BTV-R is the following (a10) or (a11) or (a12):

(a10) sequence of the single stranded DNA molecule shown in SEQ ID NO: 4;

(a11) sequence of the single stranded DNA molecule shown is SEQ ID NO: 4 from the 5′ end of the 20th to 37th nucleotides;

(a12) a DNA molecule having the same function as (a10) or (a11) by substituting and/or deleting and/or adding one or several nucleotides;

The primer pair III is composed of the primer VSV_F and the primer VSV-R;

The primer VSV-F is the following (a13) or (a14) or (a15):

(a13) sequence of the single-stranded DNA molecule shown in SEQ ID NO: 5;

(a14) sequence of the single-stranded DNA molecule shown in SEQ ID NO: 5 from the 5′ end of the 19th to 38th nucleotides;

(a15) a DNA molecule having the same function as (a13) or (a14) by substituting and/or deleting and/or adding one or more nucleotides;

The primer VSV-R is the following (a16) or (a17) or (a18);

(a16) sequence of the single stranded DNA molecule shown in SEQ ID NO: 6;

(a17) sequence of the single stranded DNA molecule shown in SELF ID NO: 6 from the 5′ end of the 20th to 38th nucleotides;

(a18) a DNA molecule having the same function as (a16) or (a17) by substituting and/or deleting and/or adding one or more nucleotides

The primer pair IV is composed of the primer BVDV-F and the primer BVDV-R;

The primer BVDV-F is the following (a19) or (a20) or (a21):

(a19) sequence of the single-stranded DNA molecule shown in SEQ ID NO: 7;

(a20) sequence of the single-stranded DNA molecule shown in SEQ ID NO: 7 from the 5′ end of the 19th to 36th nucleotides;

(a21) a DNA molecule having the same function as (a19) or (a20) by substituting and/or deleting and/or adding one or more nucleotides;

The primer BVDV-R is the following (a22) or (a23) or (a24):

(a22) sequence the single stranded DNA molecule shown in SEQ ID NO: 8;

(a23) sequence the single stranded DNA molecule shown in SEQ ID NO: 8 from the 5′ end of the 20th to 44th nucleotides;

(a24) a DNA molecule having the same function as (a22) or (a23) by substituting and/or deleting and/or adding one or several nucleotides;

The primer pair V is composed of the primer BRV-F and the primer BRV-R;

The primer BRV-F is the following (a25) or (a26) or (a27);

(a25) sequence of the single-stranded DNA molecule shown in SEQ ID NO: 9;

(a26) sequence of the single-stranded DNA molecule shown in SEQ ID NO: 9 from the 5′ end of the 19th to 40th nucleotides;

(a27) a DNA molecule having the same function as (a25) or (a26) by substituting and/or deleting and/or adding one or more nucleotides;

The primer BRV-R is the following (a28) or (a29) or (a30);

(a28) sequence the single stranded DNA molecule shown in SEQ ID NO: 10;

(a29) sequence the single stranded DNA molecule shown in SEQ ID NO: 10 from the 5′ end of the 20th to 37th nucleotides;

(a30) a DNA molecule having the same function as (a28) or (a29) by substituting and/or deleting and/or adding one or several nucleotides;

The primer pair VI is composed of the primer ETEC-F and the primer ETEC-R;

The primer ETEC-F is the following (a31) or (a32) or (a33):

(a31) sequence of the single-stranded DNA molecule shown in SEQ ID NO: 11;

(a32) sequence of the single-stranded DNA molecule shown in SEQ ID NO: 11 from the 5′ end the 19th to 36th nucleotides;

(a33) a DNA molecule having the same function as (a31) or (a32) by substituting and/or deleting and/or adding one or more nucleotides;

The primer ETEC-R is the following (a34) or (a35) or (a36):

(a34) sequence of the single stranded DNA molecule shown in SEQ ID NO: 12;

(a35) sequence of the single stranded DNA molecule shown in SEQ ID NO: 12 from the 5′ end of the 20th to 40th nucleotides;

(a36) a DNA molecule having the same function as (a34) or (a35) by substituting and/or deleting and/or adding one or more nucleotides;

The primer pair VII is composed of the primer IBRV-F and the primer IBRV-R;

The primer IBRV-F is the following (a37) or (a38) or (a39);

(a37) sequence of the single-stranded DNA molecule shown in SEQ ID NO: 13;

(a38) sequence of the single-stranded DNA molecule shown in SEQ ID NO: 13 from the 5′ end of the 19th to 41th nucleotides;

(a39) a DNA molecule having the same function as (a37) or (a38) by substituting and/or deleting and/or adding one or more nucleotides;

The primer IBRV-R is the following (a40) or (a41) or (a42):

(a40) sequence the single stranded DNA molecule shown in SEQ ID NO: 14;

(a41) sequence the single stranded DNA molecule shown in SEQ ID NO: 14 from the 5′ end of the 20th to 36th nucleotides;

(a42) a DNA molecule having the same function as (a40) or (a41) by substituting and/or deleting and/or adding one or several nucleotides;

The primer pair VIII is composed of the primer PPRV-F and the primer PPRV-R;

The primer PPRV-F is the following (a43) or (a44) or (a45):

(a43) sequence of the single-stranded DNA molecule shown in SEQ ID NO: 15;

(a44) sequence of the single-stranded DNA molecule shown in SEQ ID NO: 15 from the 5′ end of the 19th to 44th nucleotides;

(a45) a DNA molecule having the same function as (a43) or (a44) by substituting and/or deleting and/or adding one or more nucleotides;

The primer PPRV-R is the following (a46) or (a47) or (a48):

(a46) sequence the single stranded DNA molecule shown in SEQ ID NO: 16;

(a47) sequence the single stranded DNA molecule shown in SEQ ID NO: 16 from the 5′ end of the 20th to 36th nucleotides;

(a48) a DNA molecule having the same function as (a46) or (a47) by substituting and/or deleting and/or adding one or several nucleotides;

The use of the primer combination is any one of the following (b1) to (b6):

(b1) indentifying eight kinds of bovine pathogens;

(b2) preparing a kit for identification of eight kinds of bovine pathogen;

(b3) detecting whether the pathogen to be tested is foot and mouth disease virus, bluetongue virus, vesicular stomatitis virus, bovine viral diarrhea virus, bovine rotavirus, enterotoxigenic E. coli, infectious bovine rhinotracheitis virus or peste des petits ruminant virus;

(b4) preparing a kit to detect whether the pathogen to be tested is foot and mouth disease virus, bluetongue virus, vesicular stomatitis virus, bovine viral diarrhea virus, bovine rotavirus, enterotoxigenic E. coli, infectious bovine rhinotracheitis virus or peste des petits ruminant virus;

(b5) detecting whether the sample to be tested contains foot and mouth disease virus and/or bluetongue virus and/or vesicular stomatitis virus and/or bovine viral diarrhea virus and/or bovine rotavirus and/or enterotoxin E. coli and/or bovine infectious rhinotracheitis virus and/or peste des petits ruminant virus;

(b6) preparing a kit to detect whether the sample to be tested contains foot and mouth disease virus and/or bluetongue virus and/or vesicular stomatitis virus and/or bovine viral diarrhea virus and/or bovine rotavirus and/or enterotoxin E. coli and/or bovine infectious rhinotracheitis virus and/or peste des petits ruminant virus.

The present disclosure also protects the use of the primer combination as any of the following (b1) to (b6):

(b1) indentifying eight kinds of bovine pathogens;

(b2) preparing a kit for identification of eight kinds of bovine pathogens;

(b3) detecting whether the pathogen to be tested is foot and mouth disease virus, bluetongue virus, vesicular stomatitis virus, bovine viral diarrhea virus, bovine rotavirus, enterotoxigenic E. coli, infectious bovine rhinotracheitis virus or peste des petits ruminant virus;

(b4) preparing a kit to detect whether the pathogen to be tested is foot and mouth disease virus, bluetongue virus, vesicular stomatitis virus, bovine viral diarrhea virus, bovine rotavirus, enterotoxigenic E. coli, infectious bovine rhinotracheitis virus or peste des petits ruminant virus;

(b5) detecting whether the sample to be tested contains foot and mouth disease virus and/or bluetongue virus and/or vesicular stomatitis virus and/or bovine viral diarrhea virus and/or bovine rotavirus and/or enterotoxin E. coli and/or bovine infectious rhinotracheitis virus and/or peste des petits ruminant virus;

(b6) preparing a kit to detect whether the sample to be tested contains foot and mouth disease virus and/or bluetongue virus and/or vesicular stomatitis virus and/or bovine viral diarrhea virus and/or bovine rotavirus and/or enterotoxin E. coli and/or bovine infectious rhinotracheitis virus and/or peste des petits ruminant virus.

The present disclosure also protects a kit containing the primer combination; the use of the kit is the following (c1) or (c2) or (c3)

(c1) indentifying eight kinds of bovine pathogens;

(c2) detecting whether the pathogen to be tested is foot and mouth disease virus, bluetongue virus, vesicular stomatitis virus, bovine viral diarrhea virus, bovine rotavirus, enterotoxigenic E. coli, infectious bovine rhinotracheitis virus or peste des petits ruminant virus;

(c3) detecting whether the sample to be tested contains foot and mouth disease virus and/or bluetongue virus and/or vesicular stomatitis virus and/or bovine viral diarrhea virus and/or bovine rotavirus and/or enterotoxin E. coli and/or bovine infectious rhinotracheitis virus and/or peste des petits ruminant virus.

The present disclosure also protects the preparation method of the kit, comprising the steps of packaging the individual primers individually.

The present disclosure also protects a method for identifying eight bovine pathogens, comprising the following step (d1) or (d2):

(d1) the pathogen to be tested is conducted genomic DNA extraction operation and/or RNA extraction operation (DNA extraction and RNA extraction may be carried out in the same system and may also be separately carried out and post-mixed) to obtain nucleic acids; the nucleic acids of the pathogen to be tested containing RNA or may containing RNA are subjected to reverse transcription, and the DNA and/or cDNA obtained are used as templates, PCR amplification is conducted by using the primer combination (specifically, GeXP multiplex PCR amplification could be carried out; and the amplification product could be subjected to capillary electrophoresis), if the amplification product contained 165-167 bp DNA fragments, the pathogen is or is candidate for foot and mouth disease virus, if the amplified product contained 135-137 bp DNA fragment, the pathogen to be tested is or is candidate for bluetongue virus, if the amplified product contained 278-281 bp DNA fragment, the pathogen to be tested is or is candidate for vesicular stomatitis virus, if the amplified product contained a DNA fragment of 308-310 bp, the pathogen to be tested is or is candidate for bovine viral diarrhea virus. If the amplified product contained a DNA fragment of 211-214 bp, the pathogen to be tested is or is candidate for bovine rotavirus, and if the amplified product contained a DNA fragment of 342-345 bp, the pathogen to be tested is or is candidate for peste des petits ruminants virus, if the amplified product contained a DNA fragment of 252-254 bp, the pathogen to be tested is or is candidate for enterotoxigenic E. coli. If the amplified product contained a DNA fragment of 187-189 bp, the pathogen to be tested is or is candidate for infectious bovine rhinotracheitis virus;

(d2) detecting whether the germane DNA or cDNA of the pathogen to be detected contains the target sequence of the primer pair I, the target sequence of the primer pair II, the target sequence of the primer pair III, the target sequence of the primer pair IV, the target sequence of the primer pair V, the target sequence of the primer pair VI, the target sequence of the primer pair VII or the target sequence of the primer pair VIII, if the cDNA contained the target sequence of the primer pair I, the pathogen to be tested is or is candidate for foot-and-mouth disease virus. If the cDNA contained the target sequence of the primer pair II, the pathogen to be tested is or is candidate for bluetongue virus, and if the cDNA contained the target sequence of the primer pair III, the pathogen to be tested is or is candidate for vesicular stomatitis virus, if the cDNA contained the target sequence of the primer pair IV, the pathogen to be tested is or is candidate for bovine viral diarrhea virus, if the cDNA contained the target sequence of the primer pair V, the pathogen to be tested is or is candidate for bovine rotavirus, if the genomic DNA contained the target sequence of the primer pair VI, the pathogen to be tested is or is candidate for enterotoxigenic E. coli, if the cDNA contained the target sequence of the primer pair VII, the pathogen to be tested is or is candidate for infectious bovine rhinotracheitis virus, and if the cDNA contained the target sequence of the primer pair VIII, the pathogen to be tested is or is candidate for peste des petits ruminants virus.

The present disclosure also provides a method for detecting whether a pathogen to be tested is foot and mouth disease virus, bluetongue virus, vesicular stomatitis virus, bovine viral diarrhea virus, bovine rotavirus, enterotoxigenic E. coli, infectious bovine rhinotracheitis virus or peste des petits ruminants virus, including the following step (e1) or (e2):

(e1) the pathogen to be tested is conducted genomic DNA extraction operation and/or RNA extraction operation (DNA extraction and RNA extraction may be carried out in the same system and may also be separately carried out and post-mixed) to obtain nucleic acids; the nucleic acids of the pathogen to be tested containing RNA or may containing RNA are subjected to reverse transcription, and the DNA and/or cDNA obtained are used as templates, PCR amplification is conducted by using the primer combination (specifically, GeXP multiplex PCR amplification could be carried out; and the amplification product could be subjected to capillary electrophoresis), if the amplification product contained 165-167 bp DNA fragments, the pathogen is or is candidate for foot and mouth disease virus, if the amplified product contained 135-137 bp DNA fragment, the pathogen to be tested is or is candidate for bluetongue virus, if the amplified product contained 278-281 bp DNA fragment, the pathogen to be tested is or is candidate for vesicular stomatitis virus, if the amplified product contained a DNA fragment of 308-310 bp, the pathogen to be tested is or is candidate for bovine viral diarrhea virus. If the amplified product contained a DNA fragment of 211-214 bp, the pathogen to be tested is or is candidate for bovine rotavirus, and if the amplified product contained a DNA fragment of 342-345 bp, the pathogen to be tested is or is candidate for peste des petits ruminants virus, if the amplified product contained a DNA fragment of 252-254 bp, the pathogen to be tested is or is candidate for enterotoxigenic E. coli. If the amplified product contained a DNA fragment of 187-189 bp, the pathogen to be tested is or is candidate for infectious bovine rhinotracheitis virus;

(e2) detecting whether the genomic DNA or cDNA of the pathogen to be detected contains the target sequence of the primer pair I, the target sequence of the primer pair II, the target sequence of the primer pair III, the target sequence of the primer pair IV, the target sequence of the primer pair V, the target sequence of the primer pair VI, the target sequence of the primer pair VII or the target sequence of the primer pair VIII, if the cDNA contained the target sequence of the primer pair I, the pathogen to be tested is or is candidate for foot-and-mouth disease virus. If the cDNA contained the target sequence of the primer pair II, the pathogen to be tested is or is candidate for bluetongue virus, and if the cDNA contained the target sequence of the primer pair III, the pathogen to be tested is or is candidate for vesicular stomatitis virus, if the cDNA contained the target sequence of the primer pair IV, the pathogen to be tested is or is candidate for bovine viral diarrhea virus, if the cDNA contained the target sequence of the primer pair V, the pathogen to be tested is or is candidate for bovine rotavirus, if the genomic DNA contains the target sequence of the primer pair VI, the pathogen to be tested is or is candidate for enterotoxigenic E. coli, if the cDNA contained the target sequence of the primer pair VII, the pathogen to be tested is or is candidate for infectious bovine rhinotracheitis virus, and if the cDNA contained the target sequence of the primer pair VIII, the pathogen to be tested is or is candidate for peste des petits ruminants virus.

The present disclosure also provides a method for detecting whether the sample to be tested contains foot and mouth disease and/or bluetongue virus and/or vesicular stomatitis virus and/or bovine viral diarrhea virus and/or bovine rotavirus and/or enterotoxigenic E. coli and/or infectious bovine rhinotracheitis virus and/or peste des petits ruminants virus, including the following step (f1) or (f2):

(f1) the sample to be tested is conducted genomic DNA extraction operation and/or RNA extraction operation (DNA extraction and RNA extraction may be carried out in the same system and may also be separately carried out and post-mixed) to obtain a nucleic acids; the nucleic acids of the pathogen to be tested containing RNA or may containing RNA are subjected to reverse transcription, and the DNA and/or cDNA obtained are used as templates, PCR amplification is conducted by using the primer combination (specifically, GeXP multiplex PCR amplification could be carried out; and the amplification product could be subjected to capillary electrophoresis), if the amplification product contained a DNA fragment of 165-167 bp, the sample to be tested contains or suspected to contain foot and mouth disease virus, if the amplified product contained a DNA fragment of 135-137 bp, the sample to be tested contains or suspected to contain bluetongue virus, if the amplified product contained a DNA fragment of 278-281 bp, the sample to be tested contains or suspected to contain vesicular stomatitis virus, if the amplified product contained a DNA fragment of 308-310 bp, the sample to be tested contains or suspected to contain bovine viral diarrhea virus. If the amplified product contained a DNA fragment of 211-214 bp, the sample to be tested contains or suspected to contain bovine rotavirus, and if the amplified product contained a DNA fragment of 342-345 bp, the sample to be tested contains or suspected to contain peste des petits ruminants virus, if the amplified product contained a DNA fragment of 252-254 bp, the sample to be tested contains or suspected to contain enterotoxigenic E. coli. If the amplified product contained a DNA fragment of 187-189 bp, the sample to be tested contains or suspected to contain infectious bovine rhinotracheitis virus;

(f2) detecting whether the genomic DNA or cDNA of the pathogen to be detected contains the target sequence of the primer pair I, the target sequence of the primer pair II, the target sequence of the primer pair III, the target sequence of the primer pair IV, the target sequence of the primer pair V, the target sequence of the primer pair VI, the target sequence of the primer pair VII or the target sequence of the primer pair VIII, if the cDNA contained the target sequence of the primer pair I, the sample to be tested contains or suspected to contain foot-and-mouth disease virus. If the cDNA contained the target sequence of the primer pair II, the sample to be tested contains or suspected to contain bluetongue virus, and if the cDNA contained the target sequence of the primer pair III, the sample to be tested contains or suspected to contain vesicular stomatitis virus, if the cDNA contained the target sequence of the primer pair IV, the sample to be tested contains or suspected to contain bovine viral diarrhea virus, if the cDNA contained the target sequence of the primer pair V, the sample to be tested contains or suspected to contain bovine rotavirus, if the genomic DNA contained the target sequence of the primer pair VI, the sample to be tested contains or suspected to contain enterotoxigenic E. coli, if the cDNA contained the target sequence of the primer pair VII, the sample to be tested contains or suspected to contain infectious bovine rhinotracheitis virus, and if the cDNA contained the target sequence of the primer pair VIII, the sample to be tested contains or suspected to contain peste des petits ruminants virus.

The present disclosure also protects the primer combination as the following (g1) or (g2):

(g1) the primer pair I or the primer pair II or the primer pair III or the primer pair IV or the primer pair V or the primer pair VI or the primer pair VII or the primer pair VIII;

(g2) a combination of any two, three, four, five, six or seven primer pairs of the primer pair I, the primer pair II, the primer pair III, the primer pair IV, the primer pair V, the primer pair VI, the primer pair VII and the primer pair VIII.

The use of the primer combination is for the identification of foot-and-mouth disease virus and/or bluetongue virus and/or vesicular stomatitis virus and/or bovine viral diarrhea virus and/or bovine rotavirus and/or enterotoxin E. coli and/or infectious bovine rhinotracheitis virus and/or peste des petits ruminants virus.

The present disclosure also protects the application of the primer combination for the identification of foot-and-mouth disease virus and/or bluetongue virus and/or vesicular stomatitis virus and/or bovine viral diarrhea virus and/or bovine rotavirus and/or enterotoxin E. coli and/or infectious bovine rhinotracheitis virus and/or peste des petits ruminants virus.

The present disclosure also protects a kit containing the primer combination: the use of the kit is to identify foot-and-mouth disease virus and/or bluetongue virus and/or vesicular stomatitis virus and/or bovine viral diarrhea virus and/or bovine rotavirus and/or enterotoxin E. coli and/or infectious bovine rhinotracheitis virus and/or peste des petits ruminants virus.

Any one of the eight kinds of bovine pathogens as described above is foot-and-mouth disease virus, bluetongue virus, vesicular stomatitis virus, bovine viral diarrhea virus, bovine rotavirus, enterotoxin E. coli, infectious bovine rhinotracheitis virus and peste des petits ruminants virus.

Any one of the pathogens to be tested as described above may be specific for FMDV type O inactivated virus, FMDV type A inactivated virus, FMDV Asia type I inactivated virus, VSV type NJ inactivated virus, VSV type IND inactivated virus, BTV type 4 inactivated virus, BTV Type 8 inactivated virus, BTV type 9 inactivated virus, BTV type 15 inactivated virus, BTV type 17 inactivated virus, BTV type 18 inactivated virus, PPRV vaccine strain, BVDV reference strain Oregon CV24 strain (type BVDV-1), BVDV reference strain NADL strain (type BVDV-1), BVDV reference strain Yak strain (type BVDV-1), BRV reference strain NCDV, BRV reference strain BRV014, IBRV virus, ETEC reference strain 1676, ETEC reference strain 1751, ETEC reference strain B41, BVDV strain GX-BVDV1, BVDV strain GX-BVDV2, BVDV strain GX-BVDV3, BVDV strain GX-BVDV4, BVDV strain GX-BVDV5, BVDV strain GX-BVD6, BVDV strain GX-BVDV7, BVDV strain GX-BVDV8BVDV strain GX-BVDV9, BVDV strain GX-BVDV10, BRV strain GX-BVDV11, BVDV strain GX-BVDV12, BVDV strain GX-BVDV13, BVDV strain GX-BVDV041, BRV strain GX-BRV-1, BRV strain GX-BRV-2, BRV strain GX-BRV-3, BRV strain GX-BRV-4, BRV strain GX-BRV-5, BRV strain GX-BRV-6, BRV strain GX-BRV-7 or BRV strain GX-BRV-8.

Any one of “the target sequence of the primer pair I” as described above may be specific for the followings (h1) or (h2) or (h3) the DNA molecules shown in SEQ ID NO: 27; (h1) the DNA molecules shown in SEQ ID NO: 27 from the 5′ end of the 19th to 146th nucleotides; (h3) the DNA molecules having 98% or more homology with that of (h1) or (h2).

Any one of “the target sequence of the primer pair II” as described above may be specific for the following (h4) or (h5) or (h6): (h4) the DNA molecules shown in SEQ ID NO: 28; (h5) the DNA molecules shown in SEQ ID NO: 28 from the 5′ end of the 19th to 117th nucleotides; (h6) the DNA molecules having 98% or more homology with that of (h4) or (h5).

Any one of “the target sequence of the primer pair III” as described above may be specific for the following (h7) or (h8) or (h9): (h7) the DNA molecules shown in SEQ ID NO: 29; (h8) the DNA molecules shown in SEQ ID NO: 29 from the 5′ end of the 19th to 259th nucleotides; (h9) the DNA molecules having 98% or more homology with that of (h7) or (h8).

Any one of “the target sequence of the primer pair IV” as described above may be specific for the following (h10) or (h11) or (h12): (h10) the DNA molecules shown in SEQ ID NO: 30; (h11) the DNA molecules shown in SEQ ID NO: 30 from the 5′ end of the 19th to 289th nucleotides; (h12) the DNA molecules having 98% or more homology with that of (h10) or (h11).

Any one of “the target sequence of the primer pair V” as described above may be specific for the following (h13) or (h14) or (h15): (h13) the DNA molecules shown in SEQ ID NO: 31; (h14) the DNA molecules shown in SEQ ID NO: 31 from the 5′ end of the 19th to 192th nucleotides; (h15) the DNA molecules having 98% or more homology with that of (h13) or (h14).

Any one of “the target sequence of the primer pair VIII” as described above may be specific for the following (h16) or (h17) or (h18): (h16) the DNA molecules shown in SEQ ID NO: 32; (h17) the DNA molecules shown in SEQ ID NO: 32 from the 5′ end of the 19th to 325th nucleotides; (h18) the DNA molecules having 98% or more homology with that of (h16) or (h17).

Any one of “the target sequence of the primer pair VI” as described above may be specific for the following (h19) or (h20) or (h21): (h19) the DNA molecules shown in SEQ ID NO: 33:, (h20) the DNA molecules shown in SEQ ID NO: 33 from the 5′ end of the 19th to 324th nucleotides; (h21) the DNA molecules having 98% or more homology with that of (h19) or (h20).

Any one of “the target sequence of the primer pair VII” as described above may be specific for the following (h22) or (h23) or (h24): (h22) the DNA molecules shown in SEQ ID NO: 34; (h23) the DNA molecules shown in SEQ ID NO: 34 from the 5′ end of the 19th to 169th nucleotides; (h24) the DNA molecules having 98% or more homology with that of (h22) or (h23).

Any of the samples to be tested as described above may be specific for the faecal swabs, eye swabs, nasal mucus swabs, anticoagulant, OP fluid (esophageal—pharyngeal secretions), blister fluid, samples of rectal mucosal tissue, samples of blister skin tissue or lymph node tissue.

In any one of the reaction systems as described above for GeXP multiplex PCR amplification, the concentration of each primer in the primer combination is as follows: the concentrations of FMDV-F and FMDV-R are both 0.2 μmol/μL, the concentrations of BTV-F and BTV-R are both 0.2 μmol/μL, the concentrations of VSV-F and VSV-R are both 0.2 μmol/μL, the concentrations of BVDV-F and BVDV-R are both 0.2 μmol/μL, the concentrations of BRV-F and BRV-R are both 0.2 μmol/μL, the concentrations of ETEC-F and ETEC-R are both 0.2 μmol/μL, the concentrations of IBRV-F and IBRV-R are both 0.2 μmol/μL, the concentrations of PPRV-F and PPRV-R are both 2 μmol/μL.

Any one of the GeXP multiplex PCR amplification reaction system (20 μL) may be specific for: template 1 μL (10-100 ng), Genome Lab GeXP Starter Kit 5>buffer 4 μL (buffer contains universal primers, the universal primers are composed of the primer A shown in SEQ ID NO: 25 and the primer B shown in SEQ ID NO: 26 of the sequence listing, wherein the 5′ end of the primer A has a label of CY5 fluorescent group, and the working concentrations of the primer A and the primer B are both 0.25 μM), MgCl₂ (25 μM) 4 μL, 1 μL of the primer mixture containing all the primers in the primer combination, DNA polymerase 10U, and make up to 20 μL with ultrapure water.

The reaction procedure for any one of the GeXP multiplex PCR amplification as described above may be specific for: pre-denaturation at 95° C. for 5 min; 94° C. for 30 seconds, 55° C. for 30 seconds, 72° C. for 30 seconds, 10 cycles; 94° C. for 30 seconds, 68° C. for 30 seconds, 72° C. for 30 seconds, 10 cycles; 94° C. for 30 seconds, 50° C. for 30 seconds, 72° C. for 30 seconds, 10 cycles; extension at 72° C. for 5 min, and complete the reaction.

The electrophoretic conditions of any one of the capillary electrophoresis as described above is: denaturation at 90° C. for 120 seconds; 2.0 KV for 30 seconds, inhalation of sample; 6.0 KV for 35 minutes to separate the sample.

The GeXP detection method established by the present disclosure can simultaneously identify eight pathogens of bovine infectious diseases of foot-and-mouth disease virus, bluetongue virus, vesicular stomatitis virus, bovine viral diarrhea virus, bovine rotavirus, enterotoxigenic E. coli, infectious bovine rhinotracheitis virus and peste des petits ruminants virus. The method has the characteristics of high throughput, high specificity and sensitivity, and can be used for the bovine epidemiological monitoring and the differential diagnosis of sudden epidemic situation, and guarantee the healthy development of cattle industry.

### BRIEF DESCRIPTION OF THE DRAWINGS

FIG. 1 is the results of multiplex PCR amplification of the samples to be tested of Example 2.

FIG. 2 is the results of multiplex PCR amplification of the mixed samples of eight kinds of bovine pathogens of Example 2.

FIG. 3 is the amplification result of multiplex PCR when the reaction system is used in 1-5 h of Example 5.

FIG. 4 is the amplification result of multiplex PCR using the mixed solution A and the mixed solution B as a template of Example 6.

### DETAILED DESCRIPTION OF THE EMBODIMENTS

The following examples are provided to facilitate a better understanding of the present disclosure, but are not intended to limit the present disclosure. Unless otherwise specified, the experimental methods in the following examples are conventional methods. Unless otherwise specified, the test materials used in the following examples are commercially available from regular biochemical reagents stores. Three replicates were set in the following examples, and the results were averaged. Due to the error of GeXP's own system, there is a ±2 bp deviation of the amplified fragment size and the theoretical value.

FMDV type O inactivated virus, FMDV type A inactivated virus, FMDV Asia type I inactivated virus, VSV type NJ inactivated virus, VSV type IND inactivated virus, BTV type 4 inactivated virus, BTV type 8 inactivated virus, BTV type 9 inactivated virus, BTV type 15 inactivated virus, BTV type 17 inactivated virus, BTV type 18 inactivated virus, PPRV vaccine strain: Reference: Qin Min, Zou Fengcai, Yang Yunqing, etc. Establishment of multiplex PCR detection method for bluetongue, foot and mouth disease, peste des petits ruminants and vesicular stomatitis (11): 18-22; [J]. Progress in veterinary Medicine, 2015, 36 (9); 18-22; donated by the Yunnan Entry-Exit Inspection and Quarantine Bureau, the public can obtain them from Guangxi Zhuang Autonomous Region Veterinary Research Institute.

BVDV reference strain Oregon CV24 strain (type BVDV-1): China institute of veterinary drug control, Item NO: AV69.

BVDV reference strain NADL strain (type BVDV-1): China institute of veterinary drug control, Item No: AV67.

BVDV reference strain Yak strain (type BVDV-1): China institute of veterinary drug control, Item No: AV68.

BRV reference strain NCDV: China institute of veterinary drug control, Item No: AV51.

BRV reference strain BRV014: China institute of veterinary drug control, Item No: AV52.

IBRV virus: China veterinary culture collection center, Item: AV21.

ETEC reference strain 1676: China institute of veterinary drug control, Item No: 212.

ETEC reference strain 1751: China institute of veterinary drug control, Item No: 214.

ETEC reference strain B41: China institute of veterinary drug control, Item No: 215.

BVDV strain GX-BVDV1, BVDV strain GX-BVDV2, BVDV strain GX-BVDV3, BVDV strain GX-BVDV4, BVDV strain GX-BVDV5, BVDV strain GX-BVDV6, BVDV strain GX-BVDV7, BVDV strain GX-BVDV8, BVDV strain GX-BVDV9, BVDV strain GX-BVDV10, BVDV strain GX-BVDV11, BVDV strain GX-BVDV12, BVDV strain GX-BVDV13, BVDV strain GX-041: Reference: Fan Q, Xie Z, Xie L, et al. A reverse transcription loop-mediated isothermal amplification method for rapid detection of bovine viral diarrhea virus[J]. Journal of Virological Methods, 2012, 186(1-2):43-48: the public can obtain them from Guangxi Zhuang Autonomous Region Veterinary Research Institute.

BRV strain GX-BRV-1, BRV strain GX-BRV-2, BRV strain GX-BRV-3, BRV strain GX-BRV-4, BRV strain GX-BRV-5, BRV strain GX-BRV-6, BRV strain GX-BRV-7, BRV strain GX-BRV-8: Reference: Xie Z, Fan Q, Liu J, et al. Reverse transcription loop-mediated isothermal amplification assay for rapid detection of Bovine Rotavirus[J]. Bmc Veterinary Research, 2012, 8(1);451-452.; the public can obtain them from Guangxi Zhuang Autonomous Region Veterinary Research Institute.

Genome Lab GeXP Starter Kit 5×buffer: which contained the primer A shown in SEQ ID NO: 25 of the sequence listing and the primer B shown in SEQ ID NO: 26 of the sequence listing, wherein the 5′ end of the primer A has a label of CY5 fluorescent group; Beckman Coulter, Inc.

Sample Buffer: Beckman Coulter, Inc., Item No: M409196.

DNA size standard kit-400 Base Pairs: Products of Beckman Coulter, Inc., Item No: 608098.

DNA polymerase: SIGMA, USA, Item No: D4184-1.5KU.

MgCl₂ (25 μM): SIGMA, USA, Item No: M8787-1.5ML.

EasyPure Viral DNA/RNA Kit: Beijing TrausGen Biotech Co., Ltd., Item No: ER201-01.

### EXAMPLE 1

### Design and Preparation of Primer Combination

A number of primers were used to identify eight kinds of bovine pathogens of FMDV, BTV, VSV, BVDV, BRV, ETEC, IBRV and PPRV. Eight pairs of specific primers were eventually used to identify eight kinds of bovine pathogens by pre-experimenting each primer, comparing the sensitivity and specificity. Each of the specific primer pairs, the forward primer, and the reverse primer were composed of a targeting segment and a universal primer segment, the universal primer segment was located at the 5′ end of the targeting segment.

The primer pairs used to identify FMDV consisted of the following two primers (5′→3′):

FMDV-F (SEQ ID NO: 1 of the Sequence Listing):
AGGTGACACTATAGAATAGCCGTGGGACCATACAGG;
FMDV-R (SEQ ID NO: 2 of the Sequence Listing):
GTACGACTCACTATAGGGAAAGTGATCTGTAGCTTGGAATCTC.

The underlined part was a universal primer segment.

The primer pairs used to identify BTV consisted of the following two primers (5′→3′):

BTV-F (SEQ ID NO: 3 of the Sequence Listing):
AGGTGACACTATAGAATAAGGGTAACTCACAGCAAACTCAA;
BTV-R (SEQ ID NO: 4 of the Sequence Listing):
GTACGACTCACTATAGGGAGAGCAGCCTGTCCATCCC.

The underlined part was a universal primer segment.

The primer pairs used to identify VSV consisted of the following two primers (5′→3′):

VSV-F (SEQ ID NO: 5 of the Sequence Listing):
AGGTGACACTATAGAATAAAACTACTGGACGGGCTTGA;
VSV-R (SEQ ID NO: 6 of the Sequence Listing):
GTACGACTCACTATAGGGATGAGATGCCCAAATGTTGC.

The underlined part was a universal primer segment.

The primer pairs used to identify BVDV consisted of the following two primers (5′→3′):

BVDV-F (SEQ ID NO: 7 of the Sequence Listing):
AGGTGACACTATAGAATAGTGAGTTCGTTGGATGGC;
BVDV-R (SEQ ID NO: 8 of the Sequence Listing):
GTACGACTCACTATAGGGATATGTTTTGTATAAGAGTTCATTTG.

The underlined part was a universal primer segment.

The primer pairs used to identify BRV consisted of the following two primers (5′→3′):

BRV-F (SEQ ID NO: 9 of the Sequence Listing):
AGGTGACACTATAGAATACAGTGGCTTCCATTAGAAGCAT;
BRV-R (SEQ ID NO: 10 of the Sequence Listing):
GTACGACTCACTATAGGGAGGTCACATCCTCTCACTA.

The underlined part was a universal primer segment.

The primer pairs used to identify ETEC consisted of the following two primers (5′→3′):

ETEC-F (SEQ ID NO: 11 of the Sequence Listing):
AGGTGACACTATAGAATACTCAGGTGCGAAAGCGTG;
ETEC-R (SEQ ID NO: 12 of the Sequence Listing):
GTACGACTCACTATAGGGACGTTGCATCGAATTAAACCAC.

The underlined part was a universal primer segment.

The primer pairs used to identify ETEC consisted of the following two primers (5′→3′):

IBRV-F (SEQ ID NO: 13 of the Sequence Listing):
AGGTGACACTATAGAATAGCGTCATTTACAAGGAGAACATC;
IBRV-R (SEQ ID NO: 14 of the Sequence Listing):
GTACGACTCACTATAGGGAATCTCGCCCATGCCCAC.

The underlined part was a universal primer segment.

The primer pairs used to identify PPRV consisted of the following two primers (5′→3′):

PPRV-F (SEQ ID NO: 15 of the Sequence Listing):
AGGTGACACTATAGAATATGGTTTGAGAACAGAGAAATAATAGA;
PPRV-R (SEQ ID NO: 16 of the Sequence Listing):
GTACGACTCACTATAGGGAGCTTGTTGCCGGGGGTC.

The underlined part was a universal primer segment.

The printer pairs used to identify FMDV were designated as primer pair I.

The primer pairs used to identify BTV were designated as primer pair II.

The primer pairs used to identify VSV were designated as primer pair III.

The primer pairs used to identify BVDV were designated as primer pair IV.

The primer pairs used to identify BRV were designated as primer pair V.

The primer pairs used to identify ETEC were designated as primer pair VI.

The primer pairs used to identify IBRV were designated as primer pair VII.

The primer pairs used to identify PPRV were designated as primer pair VIII.

Each of the above primer pairs constituted of a primer combination.

### EXAMPLE 2

### Specificity

Firstly, a single template experiment

1. The total RNA of the samples to be tested was extracted and reverse transcribed into cDNA. The samples to be tested were: FMDV type O inactivated virus, BTV type 4 inactivated virus, VSV type NJ inactivated virus, BVDV reference strain Oregon CV24 strain (type) BVDV-1), BRV reference strain NCDV, PPRV vaccine strain.

2. The genomic DNA of the samples to be tested was extracted. The samples to be tested were ETEC reference strain 1676 and IBRV virus, respectively.

3. The cDNA obtained in step 1 and the Genomic DNA obtained in step 2 were used as templates, and the primer combination of Example 1 was used for GeXP multiplex PCR.

Multiplex PCR reaction system (20 μL): Template 1 μL, Genome Lab GeXP Starter Kit 5×buffer 4 μL (buffer contained universal primers, the universal primer consisted of the primer A shown in SEQ ID NO: 25 of the Sequence Listing and the primer B shown in SEQ ID NO: 26 of the Sequence Listing, wherein the 5′ end of primer A has a label of CY5 fluorescent group, and the working concentration of primer A and primer B were both 0.25 μM), 4 μL of the MgCl₂ (25 μM), 1 μL of the primer mixture containing all the primers in the primer combination, DNA polymerase 10 U, and made up to 20 μL with ultrapure water. In the multiplex PCR reaction system, the concentration of FMDV-F and FMDV-R were both 0.2 μmol/μL, the concentrations of BTV-F and BTV-R were both 0.2 μmol/μL, the concentrations of VSV-F and VSV-R were both 0.2 μmol/μL, the concentrations of BVDV-F and BVDV-R were both 2 μmol/μL, the concentrations of BRV-F and BRV-R were both 0.2 μmol/μL, the concentrations of ETEC-F and ETEC-R were both 0.2 μmol/μL, the concentrations of IBRV-F and IBRV-R were both 0.2 μmol/μL, the concentrations of PPRV-F and PPRV-R were both 2 μmol/μL. A negative control with equal volume of water as a template was set.

When the template was each of the cDNA samples obtained in step 1, the DNA content in the 1μL template was about 100 ng;

When the template was the genomic DNA sample obtained in step 2, the DNA content in the 1 μL template was about 100 ng;

Multiplex PCR reaction procedure: pre-denaturation at 95° C. for 5 min; 94° C. for 30 seconds, 55° C. for 30 seconds, 72° C. for 30 seconds, 10 cycles; 94° C. for 30 seconds, 68° C. for 30 seconds, 72+ C. for 30 seconds, 10 cycles; 94° C. for 30 seconds, 50° C. for 30 seconds, 72° C. for 30 seconds, 10 cycles; extension at 72° C. for 5 min, and completed the reaction.

4. The multiplex PCR amplification product of step 3 was subjected to capillary electrophoresis. The specific steps were as follows: 30 μL of multiplex PCR amplification product, 38.75 μL of sample butler and 0.25 μL of DNA size standard kit-400 base pairs were mixed by vortexing and added to the loading plate, each well was added with 1 drop of paraffin to seal the liquid surface, to avoid the oxidation of formamide and sample evaporation. 180 μL of sample butler was added to each well of the buffer plate and perform capillary electrophoresis. The conditions of capillary electrophoresis: 90° C. for 120 seconds, denaturation; 2.0 KV for 30 seconds, inhalation of sample; 6.0 KV for 35 minutes, separation of the sample. PCR products of different size fragments were separated by electrophoresis, and the fragment size and signal intensity thereof were identified by detecting the fluorescent groups carried by the PCR product with the instrument. After the electrophoresis was completed, the instrument's own software Express Profiler software was used to analyze the results.

According to the results of electrophoresis, the criteria were as follows: the size of the amplified fragment of the target gene of eight kinds of bovine pathogens were respectively: FMDV: 165-167 bp, BTV: 135-137 bp, VSV: 278-281 bp, BVDV: 308-310 bp, BRV: 211-214 bp PPRV: 342-345 bp, ETEC: 252-254 bp, IBRV: 187-189 bp. Due to the error of GeXP's own system, the amplified fragment size and the theoretical value of ±2 bp deviation were the correct results.

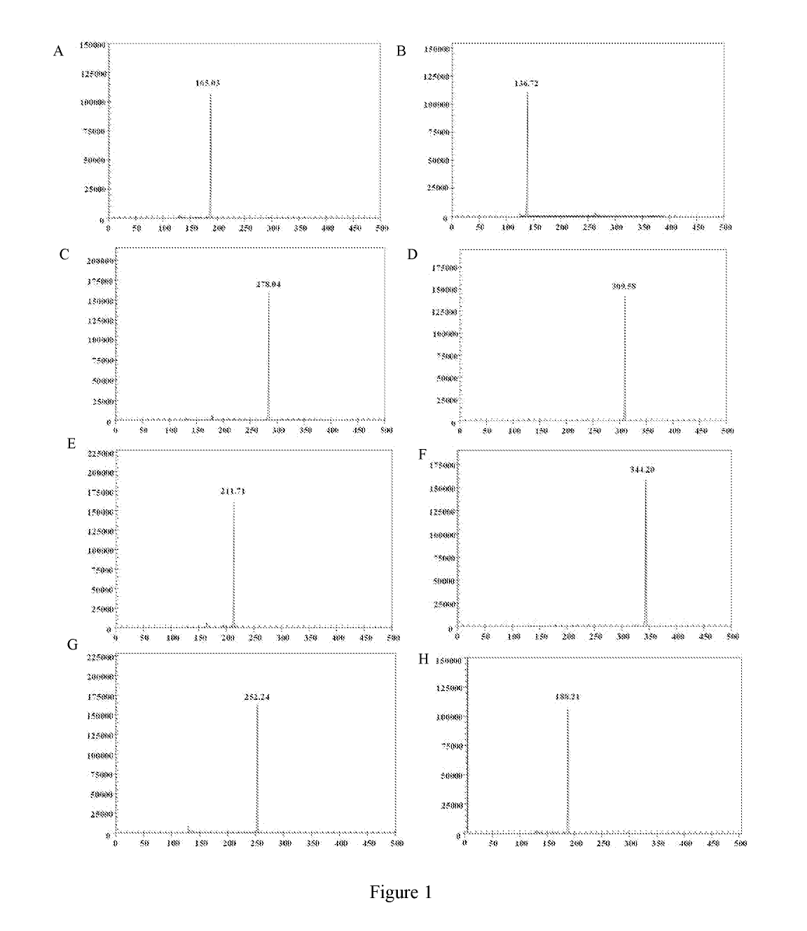

The electrophoretic results were shown in FIG. 1. In FIG. 1, the abscissa indicated the size of the fragment (in bp), and the ordinate indicated the signal intensity, namely, the content of the PCR amplified product. FIG. 1A showed the multiplex PCR amplification results of FMDV type O inactivated virus cDNA and amplified a DNA fragment of 165.03 bp. FIG. 1B showed the multiplex PCR amplification results of BTV type 4 inactivated virus cDNA and amplified a DNA fragment of 136.72 bp. FIG. 1C showed the multiplex PCR amplification results of VSV type NJ inactivated virus cDNA and amplified a DNA fragment of 278.04 bp. FIG. 1D showed the results of multiplex PCR amplification results of the BVDV reference strain Oregon CV24 strain (type BVDV-1), and amplified a DNA fragment of 309.58 bp. FIG. 1E showed the multiplex PCR amplification results of the BRV reference strain NCDV cDNA and amplified a DNA fragment of 211.71 bp. FIG. 1F showed the multiplex PCR amplification results of the PPRV vaccine strain and amplified a DNA fragment of 344.20 bp. FIG. 1G showed the multiplex PCR amplification results of the ECTC strain GX-ETEC1 genomic DNA and amplified a DNA fragment of 252.24 bp. FIG. 1H showed the multiplex PCR amplification results of the genomic DNA of IBRV virus and amplified a DNA fragment of 188.21 bp. Each reaction only showed specific singlet, no other signal peaks, and fragment size was consistent with the judgment criteria. Negative controls had no amplification and no target signal peak.

Secondly, Mixed template experiment

1. The total RNA of the samples to be tested was extracted and reverse transcribed into cDNA. The samples to be tested were: FMDV type O inactivated virus, BTV type 4 inactivated virus, VSV type NJ inactivated virus, BVDV reference strain Oregon CV24 strain (type BVDV-1), BRV reference strain NCDV, PPRV vaccine strain.

2. The genomic DNA of the samples to be tested was extracted. The samples to be tested were ETEC reference strain 1676 and IBRV virus, respectively.

3. 6 kinds of cDNA obtained in step 1 were mixed with 2 kinds of the genomic DNA obtained in step 2.

4. The mixed solutions obtained in step 3 were used as templates, and the primer combination of Example 1 was used for GeXP multiplex PCR.

Multiplex PCR reaction system (20 μL): Template 1 μL, Genome Lab GeXP Starter Kit 5×buffer 4 μL (buffer contained universal primers, the universal primer consisted of the primer A shown in SEQ ID NO: 25 of the Sequence Listing and the primer B shown in SEQ ID NO: 26 of the Sequence Listing, wherein the 5′ end of primer A has a label of CY5 fluorescent group, and the working concentration of primer A and primer B were both 0.25 μM), 4 μL of the MgCl₂ (25 μM), 1 μL of the primer mixture containing all the primers in the primer combination, DNA polymerase 10 U, and made up to 20 μL with ultrapure water. In the multiplex PCR reaction system, the concentration of FMDV-F and FMDV-R were both 0.2 μmol/μL, the concentrations of BTV-F and BTV-R were both 0.2 μmol/μL, the concentrations of VSV-F and VSV-R were both 0.2 μmol/μL, the concentrations of BVDV-F and BVDV-R were both 2 μmol/μL, the concentrations of BRV-F and BRV-R were both 0.2 μmol/μL, the concentrations of ETEC-F and ETEC-R were both 0.2 μmol/μL, the concentrations of IBRV-F and IBRV-R were both 0.2 μmol/μL, the concentrations of PPRV-F and PPRV-R were both 2 μmol/μL. A negative control with equal volume of water as a template was set.

In the 1 μL template, the cDNA of the FMDV type O inactivated virus was about 100 ng, the cDNA of the BTV type 4 inactivated virus was about 100 ng, the cDNA of the VSV type NJ inactivated virus was about 100 ng, the cDNA of the BVDV reference strain Oregon CV24 strain (BVDV-1) was about 100 ng, the genomic DNA of the BRV reference strain NCDV was about 100 ng, the genomic DNA of the PPRV vaccine strain was about 100 ng, the genomic DNA of the ECTC strain GX-ETEC1 was about 100 ng, the genomic DNA of the IBRV virus was about 100 ng.

Multiplex PCR reaction procedure: pre-denaturation at 95° C. for 5 min; 94° C. for 30 seconds, 55° C. for 30 seconds, 72° C. for 30 seconds, 10 cycles; 94° C. for 30 seconds, 68° C. for 30 seconds, 72° C. for 30 seconds, 10 cycles, 94° C. for 30 seconds, 50° C. for 30 seconds, 72° C. for 30 seconds, 10 cycles; extension at 72° C. for 5 min, and completed the reaction.

5. The multiplex PCR amplification product of step 4 was subjected to capillary electrophoresis. The specific steps were as follows: 3 μL of multiplex PCR amplification product, 38.75 μL of sample buffer and 0.25 μL of DNA size standard kit-400 base pairs were mixed by vortexing and added to the loading plate, each well was added with 1 drop of paraffin to seal the liquid surface, to avoid the oxidation of formamide and sample evaporation. 180 μL of sample buffer was added to each well of the buffer plate and perform capillary electrophoresis. The conditions of capillary electrophoresis: 90° C. for 120 seconds, denaturation; 2.0 KV for 30 seconds, inhalation of sample; 6.0 KV for 35 minutes, separation of the sample. PCR products of different size fragments were separated by electrophoresis, and the fragment size and signal intensity thereof were identified by detecting the fluorescent groups carried by the PCR product with the instrument. After the electrophoresis was completed, the instrument's own software Express Profiler software was used to analyze the results.

According to the results of electrophoresis, the criteria were as follows: the size of the amplified fragment of the target gene of eight kinds of bovine pathogens were respectively: FMDV: 165-167 bp, BTV: 135-137 bp, VSV: 278-281 bp, BVDV: 308-310 bp, BRV: 211-214 bp, PPRV: 342-345 bp, ETEC: 252-254 bp, IBRV: 187-189 bp.

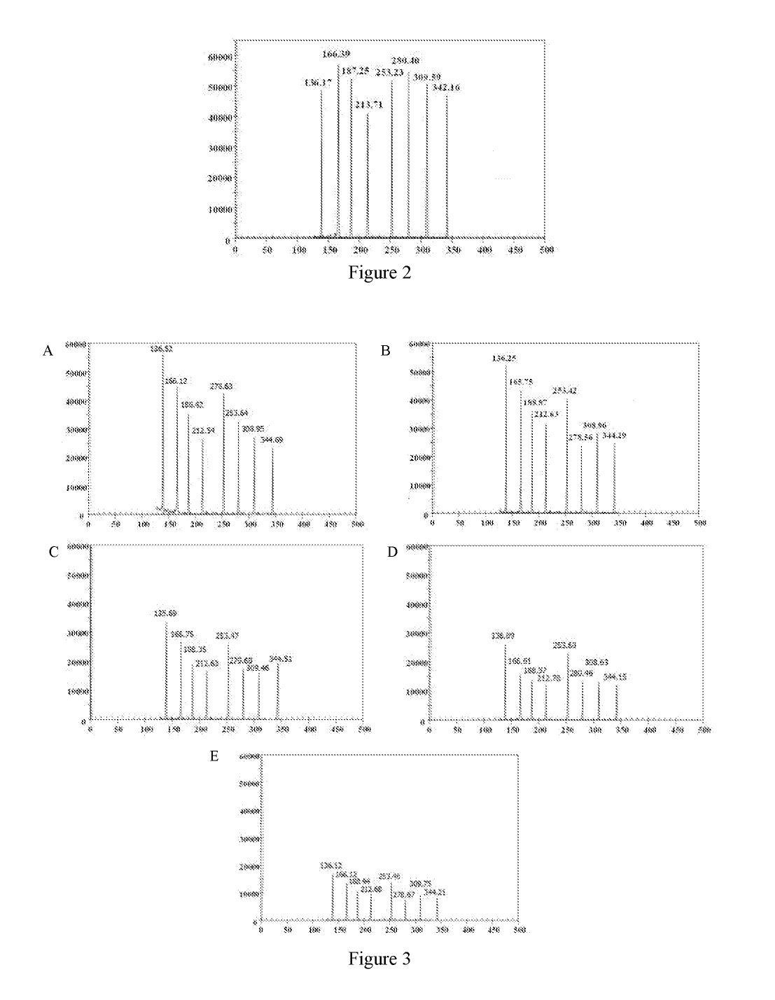

The electrophoretic results were shown in FIG. 2. In FIG. 2, the abscissa indicated the size of the fragment (in bp), and the ordinate indicated the signal intensity, namely, the content of the PCR amplified product. The results showed that eight signal peaks corresponding to eight pathogens could be detected by GeXP multiplex PCR simultaneously, FMDV: 166.39 bp, BTV: 136.17 bp, VSV: 280.40 bp, BVDV: 309.59 bp, BRV: 213.71 bp, PPRV: 342.16 bp, ETEC: 253.23 bp, IBRV: 187.25 bp, no other peaks. Negative controls had no amplification and no target signal peak.

### EXAMPLE 3

### Universality

1. The total RNA of the samples to be tested was extracted and reverse transcribed into cDNA. The samples to be tested were: the FMDV type O inactivated virus, FMDV type A inactivated virus, FMDV Asia type I inactivated virus, BTV type 4 inactivated virus, BTV Type 8 inactivated virus, BTV type 9 inactivated virus, BTV type 15 inactivated virus, BTV type 17 inactivated virus, BTV type 18 inactivated virus, VSV type NJ inactivated virus, VSV type IND inactivated virus, BVDV reference strain Oregon CV24 strain (type BVDV-1), BVDV reference strain NADL strain (type BVDV-1), BVDV reference strain Yak strain (type BVDV-1), BVDV strain GX-BVDV1, BVDV strain GX-BVDV2BVDV strain GX-BVDV3, BVDV strain GX-BVDV4, BVDV strain GX-BVDV5, BVDV strain GX-BVD6, BVDV strain GX-BVDV7, BVDV strain GX-BVDV8, BVDV strain GX-BVDV9, BVDV strain GX-BVDV10, BRV strain GX-BVDV11, BVDV strain GX-BVDV12, BVDV strain GX-BVDV13, BVDV strain GX-BVDV041, BRV reference strain NCDV, BRV reference strain BRV014, BRV strain GX-BRV-1, BRV strain GX-BRV-2, BRV strain GX-BRV-3, BRV strain GX-BRV-4, BRV strain GX-BRV-5, BRV strain GX-BRV-6, BRV strain GX-BRV-7, BRV strain GX-BRV-8, PPRV vaccine strain.

2. The genomic DNA of the samples to be tested was extracted. The samples to be tested were ETEC reference strain 1676, ETEC reference strain B41 and IBRV virus, respectively.

3. The cDNA obtained in step 1 and the genomic DNA obtained in Step 2 were used as templates, and the primer combination of Example 1 was used for GeXP multiplex PCR.

Multiplex PCR reaction system (20 μL): Template 1 μL, Genome Lab GeXP Starter Kit 5×buffer 4 μL (buffer contained universal primers, the universal primer consisted of the primer A shown in SEQ ID NO: 25 of the Sequence Listing and the primer B shown in SEQ ID NO: 26 of the Sequence Listing, wherein the 5′ end of primer A has a label of CY5 fluorescent group, and the working concentration of primer A and primer B were both 0.25 μM), 4 μL of the MgCl₂ (25 μM), 1 μL of the primer mixture containing all the primers in the primer combination, DNA polymerase 10 U, and made up to 20 μL with ultrapure water. In the multiplex PCR reaction system, the concentration of FMDV-F and FMDV-R were both 0.2 μmol/μL, the concentrations of BTV-F and BTV-R were both 0.2 μmol/μL, the concentrations of VSV-F and VSV-R were both 0.2 μmol/μL, the concentrations of BVDV-F and BVDV-R were both 2 μmol/μL, the concentrations of BRV-F and BRV-R were both 0.2 μmol/μL, the concentrations of ETEC-F and ETEC-R were both 0.2 μmol/μL, the concentrations of IBRV-F and IBRV-R were both 0.2 μmol/μL, the concentrations of PPRV-F and PPRV-R were both 2 μmol/μL. A negative control with equal volume of water as a template was set.

When the template was each of the cDNA samples obtained in step 1, the DNA content in the 1 μL template was about 100 ng;

When the template was the genomic DNA sample obtained in step 2, the DNA content in the 1 μL template was about 100 ng;

Multiplex PCR reaction procedure: pre-denaturation at 95° C. for 5 min; 94° C. for 30 seconds, 55° C. for 30 seconds, 72° C. for 30 seconds, 10 cycles; 94° C. for 30 seconds, 68° C. for 30 seconds, 72° C. for 30 seconds, 10 cycles; 94° C. for 30 seconds, 50° C. for 30 seconds, 72° C. for 30 seconds, 10 cycles; extension at 72° C. for 5 min, and completed the reaction.

4. The multiplex PCR amplification product of step 3 was subjected to capillary electrophoresis. The specific steps were as follows: 3 μL of multiplex PCR amplification product. 38.75 μL of sample buffer and 0.25 μL of DNA size standard kit-400 base pairs were mixed by vortexing and added to the loading plate, each well was added with 1 drop of paraffin to seal the liquid surface, to avoid the oxidation of formamide and sample evaporation. 180 μL of sample buffer was added to each well of the buffer plate and perform capillary electrophoresis. The conditions of capillary electrophoresis: 90° C. for 120 seconds, denaturation; 2.0 KV for 30 seconds, inhalation of sample; 6.0 KV for 35 minutes, separation of the sample. PCR products of different size fragments were separated by electrophoresis, and the fragment size and signal intensity thereof were identified by detecting the fluorescent groups carried by the PCR product with the instrument. After the electrophoresis was completed, the instrument's own software Express Profiler software was used to analyze the results.

According to the results of electrophoresis, the criteria were as follows: the size of the amplified fragment of the target gene of eight kinds of bovine pathogens were respectively: FMDV: 165-167 bp, BTV: 135-137 bp, VSV: 278-281 bp, BVDV: 308-310 bp, BRV: 211-214 bp, PPRV: 342-345 bp, ETEC: 252-254 bp, IBRV: 187-189 bp. Due to the error of GeXP's own system, the amplified fragment size and the theoretical value of ±2 bp deviation were the correct results.

The samples to be tested were performed the multiplex PCR amplification with the primer combination of Example 1. Each sample only showed the specific single peaks of the corresponding pathogens and no other signal peaks, and the fragment size was consistent with the criterion. The results showed that the primer combination designed in Example 1 was generally applicable to eight kinds of bovine pathogens.

### EXAMPLE 4

### Preparation of Plasmid Standards

The target fragment of each strain was amplified by using the specific primers designed in the present disclosure, and the obtained fragment was ligated with the pEASY-T1 vector to construct a recombinant plasmid of each of the target fragments of GeXP (the double-stranded DNA molecule corresponding to the SEQ ID NO: 17 of the sequence listing was ligated with the pEASY-T1 vector to obtain the recombinant plasmid pEASY-T1-FMDV; the double-stranded DNA molecule corresponding to the SEQ ID NO: 18 of the sequence listing was ligated with the pEASY-T1 vector to obtain the recombinant plasmid pEASY-T1-BTV; the double-stranded DNA molecule corresponding to the SEQ ID NO: 19 of the sequence listing was ligated with pEASY-T1 vector to obtain recombinant plasmid pEASY-T1-VSV; the double-stranded DNA molecule corresponding to the SEQ ID NO: 20 of the sequence listing was ligated with pEASY-T1 vector to obtain recombinant plasmid pEASY-T1-BVDV; the double-stranded DNA molecule corresponding to the SEQ ID NO: 21 of the sequence listing was ligated with the pEASY-T1 vector to obtain the recombinant plasmid pEASY-T1-BRV; the double-stranded DNA molecule corresponding to the SEQ ID NO: 22 of the sequence listing was ligated with the pEASY-T1 vector to obtain the recombinant plasmid pEASY-T1-PPRV; the double-stranded DNA molecule corresponding to the SEQ ID NO: 23 of the sequence listing was ligated with the pEASY-T1 vector to obtain the recombinant plasmid pEASY-T1-ETEC; the double-stranded DNA molecule corresponding to the SEQ ID NO: 24 of the sequence listing was ligated with the pEASY-T1 vector to obtain the recombinant plasmid pEASY-T1-IBRV), the positive clone plasmid was extracted. The concentrations of plasmids of pEASY-T1-IBRV and pEASY-T1-ETEC were determined with NanoDrop UV spectrophotometer. The positive clones were extracted (pEASY-T1-BTV, pEASY-T1-FMDV, pEASY-T1-BRV, pEASY-T1-VSV, pEASY-T1-BVDV and pEASY-T1-PPRV) and transcripted into RNA in vitro with reference to the T7 in vitro transcription kit instructions. RNA concentration was measured with NanoDrop. The standard of copy number was calculated according to the molecular weight and concentration. The concentration of each plasmid was diluted to 10^{x} copies/μl-1 copies/μl, namely, obtained the FMDV standard, the BTV standard, the VSV standard, the BVDV standard, the BRV standard, the PPRV standard, the ETEC standard and the IBRV standard, saved at −70° C. standby.

### EXAMPLE 5

### Sensitivity

1. The FMDV standard, the BTV standard, the VSV standard, the BVDV standard, the BRV standard, the ETEC standard, the IBRV standard, and the PPRV standard prepared in Example 4 were mixed to obtain a mixed solution.

2. The mixed solution obtained in step 2 was diluted with a 10-fold gradient of ddH2O to obtain each diluent.

3. The dilution obtained in step 2 was used as a template, and the primer combination prepared in Example 1 was subjected to GeXP multiplex PCR.

Multiplex PCR reaction system (20 μL): Template 1 μL, Genome Lab GeXP Starter Kit 5×buffer 4 μL (buffer contained universal primers, the universal primer consisted of the primer A shown in SEQ ID NO: 25 of the Sequence Listing and the primer B shown in SEQ ID NO: 26 of the Sequence Listing, wherein the 5′ end of primer A has a label of CY5 fluorescent group, and the working concentration of primer A and primer B were both 0.25 μM), 4 μL of the MgCl₂ (25 μM), 1 μL of the primer mixture containing all the primers in the primer combination, DNA polymerase 10 U, and made up to 20 μL with ultrapure water. In the multiplex PCR reaction system, the concentration of FMDV-F and FMDV-R were both 0.2 μmol/μL, the concentrations of BTV-F and BTV-R were both 0.2 μmol/μL, the concentrations of VSV-F and VSV-R were both 0.2 μmol/μL, the concentrations of BVDV-F and BVDV-R were both 2 μmol/μL, the concentrations of BRV-F and BRV-R were both 0.2 μmol/μL, the concentrations of ETEC-F and ETEC-R were both 0.2 μmol/μL, the concentrations of IBRV-F and IBRV-R were both 0.2 μmol/μL, the concentrations of PPRV-F and PPRV-R were both 2 μmol/μL. A negative control with equal volume of water as a template was set.

Due to the different dilution of the diluent used, the following different reaction systems were formed:

In the reaction system 1, the initial concentrations of FMDV standard, BTV standard, VSV standard, BVDV standard, BRV standard, ETEC standard, IBRV standard and PPRV standards were 10⁶ copies/μL.

In the reaction system 2, the initial concentrations of FMDV standard, BTV standard, VSV standard, BVDV standard, BRV standard, ETEC standard, IBRV standard and PPRV standards were 10⁵ copies/μL;

In the reaction system 3, the initial concentrations of FMDV standard, BTV standard, VSV standard, BVDV standard, BRV standard, ETEC standard, IBRV standard and PPRV standards were 10⁴ copies/μL;

In the reaction system 4, the initial concentrations of FMDV standard, BTV standard, VSV standard, BVDV standard, BRV standard, ETEC standard, IBRV standard and PPRV standards were 10³ copies/μL;

In the reaction system 5, the initial concentrations of FMDV standard, BTV standard, VSV standard, BVDV standard, BRV standard, ETEC standard, IBRV standard and PPRV standards were 10² copies/μL:

In the reaction system 6, the initial concentrations of FMDV standard, BTV standard, VSV standard, BVDV standard, BRV standard, ETEC standard, IBRV standard and PPRV standards were 10 copies/μL;

In the reaction system 7, the initial concentrations of FMDV standard, BTV standard, VSV standard, BVDV standard, BRV standard, ETEC standard, IBRV standard and PPRV standards were 1 copy/μL;

Multiplex PCR reaction procedure: pre-denaturation at 95° C. for 5 min; 94° C. for 30 seconds, 55° C. for 30 seconds, 72° C. for 30 seconds, 10 cycles; 94° C. for 30 seconds, 68° C. for 30 seconds, 72° C. for 30 seconds, 10 cycles; 94° C. for 30 seconds, 50° C. for 30 seconds, 72° C. for 30 seconds, 10 cycles; extension at 72° C. for 5 min, and completed the reaction.

4. The multiplex PCR amplification product of step 3 was subjected to capillary electrophoresis. The specific steps were as follows: 3 μL of multiplex PCR amplification product, 38.75 μL, of sample buffer and 0.25 μL of DNA size standard kit-400 base pairs were mixed by vortexing and added to the loading plate, each well was added with 1 drop of paraffin to seal the liquid surface, to avoid the oxidation of formamide and sample evaporation. 180 μL of sample buffer was added to each well of the buffer plate and perform capillary electrophoresis. The conditions of capillary electrophoresis: 90° C. for 120 seconds, denaturation; 2.0 KV for 30 seconds, inhalation of sample: 6.0 KV for 35 minutes, separation of the sample. PCR products of different size fragments were separated by electrophoresis, and the fragment size and signal intensity thereof were identified by detecting the fluorescent groups carried by the PCR product with the instrument. After the electrophoresis was completed, the instrument's own software Express Profiler software was used to analyze the results.

According to the results of electrophoresis, the criteria were as follows: the size of the amplified fragment of the target gene of eight kinds of bovine pathogens were respectively: FMDV: 165-167 bp, BTV: 135-137 bp, VSV: 278-281 bp, BVDV: 308-310 bp, BRV: 211-214 bp, PPRV: 342-345 bp, ETEC: 252-254 bp, IBRV: 187-189 bp. Due to the error of GeXP's own system, the amplified fragment size and the theoretical value of ±2 bp deviation were the correct results.

The electrophoretic results were shown in FIG. 3. FIGS. 3A-3E sequentially corresponded to the amplification results of the multiplex PCR when using the reaction system 1-5. In FIG. 3, the abscissa indicated the size of the fragment (in bp), and the ordinate indicated the signal intensity, namely, the content of the PCR amplified product. The results showed that when the concentration of DNA to be tested in the assay system was as low as 100 copies/μL, the eight pathogens could also be detected.

### EXAMPLE 6

### Interference

1. The IBRV standard, the BRV standard, the ETEC standard, the BVDV standard, and the PPRV standard prepared in Example 4 were mixed to obtain a mixed solution A.

2. The FMDV standard, the BTV standard, the IBRV standard, the BRV standard, and the ETEC standard prepared in Example 4 were mixed to obtain a mixed solution B.

3. The mixed solution A obtained in step 1 and the mixed solution B obtained in step 2 were used as templates respectively, and the primer combination prepared in Example 1 was subjected to GeXP multiplex PCR.

Multiplex PCR reaction system (20 μL): Template 1 μL, Genome GeXP Starter Kit 5×buffer 4μL (buffer contained universal primers, the universal primer consisted of the primer A shown in SEQ ID NO: 25 of the Sequence Listing and the primer B shown in SEQ ID NO: 26 of the Sequence Listing, wherein the 5′ end of primer A has a label of CY5 fluorescent group, and the working concentration of primer A and primer B were both 0.25 μM), 4 μL of the MgCl₂ (25 μM), 1 μL of the primer mixture containing all the primers in the primer combination, DNA polymerase 10 U, and made up to 20 μL with ultrapure water. In the multiplex PCR reaction system, the concentration of FMDV-F and FMDV-R were both 0.2 μmol/μL, the concentrations of BTV-F and BTV-R were both 0.2 μmol/μL, the concentrations of VSV-F and VSV-R were both 0.2 μmol/μL, the concentrations of BVDV-F and BVDV-R were both 2 μmol/μL, the concentrations of BRV-F and BRV-R were both 0.2 μmol/μL, the concentrations of ETEC-F and ETEC-R were both 0.2 μmol/μL, the concentrations of IBRV-F and IBRV-R were both 0.2 μmol/μL, the concentrations of PPRV-F and PPRV-R were both 2 μmol/μL. A negative control with equal volume of water as a template was set.

When the mixed solution A was template, in the reaction system, the concentration of IBRV standard was 10³ copies/μL, the concentration of BRV standard was 10⁵ copies/μL, the concentration of ETEC standard was 10⁵ copies/μL, the concentration of BVDV standard was 10⁵ copies/μL, the concentration of PPRV standard was 10⁷ copies/μL.

When the mixed solution B was template, in the reaction system, the concentration of FMDV standard was 10⁴ copies/μL, the concentration of BTV standard was 10⁸ copies/μL, the concentration of IBRV standard was 10⁴ copies/μL, the concentration of BRV standard was 10⁵ copies/μL, the concentration of ETEC standard was 10⁵ copies/μL.

Multiplex PCR reaction procedure: pre-denaturation at 95° C. for 5 min; 94° C. for 30 seconds, 55° C. for 30 seconds, 72° C. for 30 seconds, 10 cycles; 94° C. for 30 seconds, 68° C. for 30 seconds, 72° C. for 30 seconds, 10 cycles; 94° C. for 30 seconds, 50° C. for 30 seconds, 72° C. for 30 seconds, 10 cycles; extension at 72° C. for 5 min, and completed the reaction.

4. The multiplex PCR amplification product of step 3 was subjected to capillary electrophoresis. The specific steps were as follows: 3 μL of multiplex PCR amplification product, 38.75 μL of sample buffer and 0.25 μL of DNA size standard kit-400 base pairs were mixed by vortexing and added to the loading plate, each well was added with 1 drop of paraffin to seal the liquid surface, to avoid the oxidation of formamide and sample evaporation. 180 μL of sample buffer was added to each well of the buffer plate and perform capillary electrophoresis. The conditions of capillary electrophoresis: 90° C. for 120 seconds, denaturation; 2.0 KV for 30 seconds, inhalation of sample; 6.0 KV for 35 minutes, separation of the sample. PCR products of different size fragments were separated by electrophoresis, and the fragment size and signal intensity thereof were identified by detecting the fluorescent groups carried by the PCR product with the instrument. After the electrophoresis was completed, the instrument's own software Express Profiler software was used to analyze the results.

According to the results of electrophoresis, the criteria were as follows: the size of the amplified fragment of the target gene of eight kinds of bovine pathogens were respectively: FMDV: 165-167 bp, BTV: 135-137 bp, VSV: 278-281 bp, BVDV: 308-310 bp, BRV: 211-214 bp, PPRV: 342-345 bp, ETEC: 252-254 bp, IBRV: 187-189 bp. Due to the error of GeXP's own system, the amplified fragment size and the theoretical value of ±2 bp deviation were the correct results.

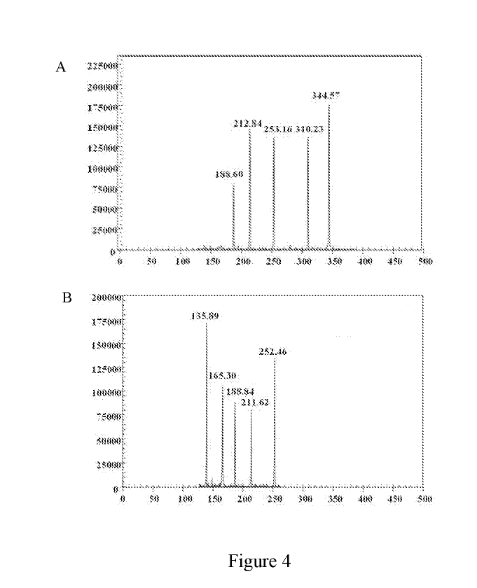

The electrophoretic results were shown in FIG. 4. In FIG. 4, the abscissa indicated the size of the fragment (in bp), and the ordinate indicated the signal intensity, namely, the content of the PCR amplified product. FIG. 4A showed the results of multiplex PCR by using the mixed solution A as a template, and FIG. 4B showed the results of multiplex PCR by using the mixed solution B as a template. The results showed that when the concentrations of the starting template in the reaction system was greatly different, the template with low concentration could still be detected accurately and sensitively, and the interference was small.

### EXAMPLE 7

### Clinical Sample Testing

The samples to be tested were: 305 clinical samples, including 156 fecal swabs, 30 eye swabs, 30 nasal mucus swabs, 70 anticoagulant, 2 OP fluid (esophageal-pharyngeal secretions), 2 blister fluid, 15 tissue samples (10 rectal mucosa, 2 blister skin, 3 lymph nodes). Clinical samples were collected around in Guangxi from 2012 to 2014, about ½ of the samples were derived from around dairy cows without symptoms, about ¼ of the samples were derived from cattle with clinical symptoms such as diarrhea, weight loss, and runny nose, ¼ of the samples were derived from cattle with the low spirit, swallowing difficulties, fever, oral erosion of blisters, white foam in nose and mouth.

1. The DNA/RNA of the sample to be tested was extracted by using the EasyPure Viral DNA/RNA Kit to obtain a DNA/RNA mixed solution.

2. The DNA/RNA mixed solution obtained in step 1 was reverse-transcribed to obtain a DNA/cDNA mixed solution.

3. The DNA/cDNA mixed solution obtained in step 2 was used as a template, and the primer combination prepared in Example 1 was subjected to GeXP multiplex PCR.

Multiplex PCR reaction system (20 μL): Template 1 μL (DNA content of 10-100 ng), Genome Lab GeXP Starter Kit 5×buffer 4 μL (buffer contained universal primers, the universal primer consisted of the primer A shown in SEQ ID NO: 25 of the Sequence Listing and the primer B shown in SEQ ID NO: 26 of the Sequence Listing, wherein the 5′ end of primer A has a label of CY5 fluorescent group, and the working concentration of primer A and primer B were both 0.25 μM), 4 μL of the MgCl₂ (25 μM), 1 μL of the primer mixture containing all the primers in the primer combination, DNA polymerase 10 U, and made up to 20 μL with ultrapure water. In the multiplex PCR reaction system, the concentration of FMDV-F and FMDV-R were both 0.2 μmol/μL, the concentrations of BTV-F and BTV-R were both 0.2 μmol/μL, the concentrations of VSV-F and VSV-R were both 0.2 μmol/μL, the concentrations of BVDV-F and BVDV-R were both 2 μmol/μL, the concentrations of BRV-F and BRV-R were both 0.2 μmol/μL, the concentrations of ETEC-F and ETEC-R were both 0.2 μmol/μL, the concentrations of IBRV-F and IBRV-R were both 0.2 μmol/μL, the concentrations of PPRV-F and PPRV-R were both 2 μmol/μL. A negative control with equal volume of water as a template was set.

Multiplex PCR reaction procedure: pre-denaturation at 95° C. for 5 min; 94° C. for 30 seconds, 55° C. for 30 seconds, 72° C. for 30 seconds. 10 cycles; 94° C. for 30 seconds, 68° C. for 30 seconds, 72° C. for 30 seconds, 10 cycles; 94° C. for 30 seconds, 50° C. for 30 seconds, 72° C. for 30 seconds, 10 cycles; extension at 72° C. for 5 min, and completed the reaction.

4. The multiplex PCR amplification product of step 3 was subjected to capillary electrophoresis. The specific steps were as follows: 3 μL of multiplex PCR amplification product, 38.75 μL of sample buffer and 0.25 μL of DNA size standard kit-400 base pairs were mixed by vortexing and added to the loading plate, each well was added with 1 drop of paraffin to seal the liquid surface, to avoid the oxidation of formamide and sample evaporation. 180 μL of sample buffer was added to each well of the buffer plate and perform capillary electrophoresis. The conditions of capillary electrophoresis: 90° C. for 120 seconds, denaturation; 2.0 KV for 30 seconds, inhalation of sample; 6.0 KV for 35 minutes, separation of the sample. PCR products of different size fragments were separated by electrophoresis, and the fragment size and signal intensity thereof were identified by detecting the fluorescent groups carried by the PCR product with the instrument. After the electrophoresis was completed, the instrument's own software Express Profiler software was used to analyze the results.

According to the results of electrophoresis, the criteria were as follows: the size of the amplified fragment of the target gene of eight kinds of bovine pathogens were respectively: FMDV: 165-167 bp, BTV: 135-137 bp, VSV: 278-281 bp, BVDV: 308-310 bp, BRV: 211-214 bp, PPRV: 342-345 bp, ETEC: 252-254 bp, IBRV: 187-189 bp. Due to the error of GeXP's own system, the amplified fragment size and the theoretical value of ±2 bp deviation were the correct results.

5. The positive amplification products obtained in step 4 were sequenced to verify the correctness of the results.

The test results were shown in Table 1.

TABLE 1
Test results statistics of the clinical samples

| | Positive Numbers | Numbers of | The proportion of |
|---|---|---|---|
| | of the GeXP | the positive | positive samples |
| Pathogens | multiplex PCR | sequences | to the total samples |
| FMDV | 6 | 6 | 2.0% |
| BTV | 32 | 32 | 10.5% |
| VSV | 0 | 0 | 0% |
| BVDV | 41 | 41 | 13.4% |
| BRV | 8 | 8 | 2.6% |
| ETEC | 55 | 55 | 18.0% |
| IBRV | 4 | 4 | 1.31% |
| PPRV | 2 | 2 | 0.7% |
| BVDV + ETEC | 23 | 23 | 10.5% |
| BRV + ETEC | 5 | 5 | 1.6% |

In the 305 samples, 148 test results were positive, of which 92 were single infections (single-infection samples accounted for 30.1% of the positive samples), and mixed infections accounted for 28 copies (mixed infection samples accounted for 9.2% of positive samples). Sequencing results showed that the positive results were all corresponding to the virus, with no specific amplification of false positives.

What is claimed is:

1. A primer combination, wherein the combination consisting of one or more primer pairs selected from the group consisting of primer pair I, primer pair II, primer pair III, primer pair IV, primer pair V, primer pair VI, primer pair VII and primer pair VIII;
wherein the primer pair I comprises a primer FMDV-F and a primer FMDV-R;
wherein the primer FMDV-F is selected from a1 or a2,
wherein a1 is a single-stranded DNA molecule having the sequence of SEQ ID NO: 1;
a2 is a single-stranded DNA molecule having the sequence of the 19th to 36th nucleotides from the 5′ end of SEQ ID NO: 1;
wherein the primer FMDV-R is selected from a4 or a5:
wherein a4 is a single stranded DNA molecule having the sequence of SEQ ID NO: 2;
a5 is a single stranded DNA molecule having the sequence of the 20th to 43th nucleotides from the 5′ end of SEQ ID NO: 2;
wherein the primer pair II comprises a primer BTV-F and a primer BTV-R;
wherein the primer BTV-F is selected from a7 or a8:
wherein a7 a single stranded DNA molecule having the sequence of SEQ ID NO: 3;
a8 is a single stranded DNA molecule having the sequence of the 19th to 41th nucleotides from the 5′ end of SEQ ID NO: 3;
wherein the primer BTV-R is selected from a10 or a11:
wherein a10 is a single stranded DNA molecule having the sequence of SEQ ID NO: 4;
a11 is a single stranded DNA molecule having the sequence of the 20th to 37th nucleotides from the 5′ end of SEQ ID NO: 4;
wherein the primer pair III comprises a primer VSV-F and a primer VSV-R;
wherein the primer VSV-F is selected from a13 or a14:
wherein a13 is a single-stranded DNA molecule having the sequence of SEQ ID NO: 5;
a14 is a single-stranded DNA molecule having the sequence of the 9th to 38th nucleotides from the 5′ end of SEQ ID NO: 5;
wherein the primer VSV-R is selected from a16 or a17:
wherein a16 is a single-stranded DNA molecule having the sequence of SEQ ID NO: 6;
a17 is a single stranded DNA molecule having the sequence of the 20th to 38th nucleotides from the 5′ end of SEQ ID NO: 6;
wherein the primer pair IV comprises a primer BVDV-F and a primer BVDV-R;
wherein the primer BVDV-F is selected from a19 or a20:
wherein a19 is a single-stranded DNA molecule having the sequence of SEQ ID NO: 7;
a20 is a single stranded DNA molecule having the sequence of the 19th to 36th nucleotides from the 5′ end of SEQ ID NO: 7;
wherein the primer BVDV-R is selected from a22 or a23:
wherein a22 is a single-stranded DNA molecule having the sequence of SEQ NO: 8;
a23 sequence is a single-stranded DNA molecule having the sequence of the 20th to 44th nucleotides from the 5′ end of SEQ ID NO: 8;
wherein the primer pair V is comprises a primer BRV-F and a primer BRV-R;
wherein the primer BRV-F is selected from a25 or a26:
wherein a25 is a single-stranded DNA molecule having the sequence of SEQ ID NO: 9;
a26 is a single-stranded DNA molecule having the sequence of the 19th to 40th nucleotides from the 5′ end of SEQ ID NO: 9;
wherein the primer BRV-R is selected from a28 or a29:
wherein a28 is a single stranded DNA molecule having the sequence of SEQ ID NO: 10;
a29 is a single stranded DNA molecule having the sequence of the 20th to 37th nucleotides from the 5′ end of SEQ ID NO: 10;
wherein the primer pair VI comprises a primer ETEC-F and a primer ETEC-R;
wherein the primer ETEC-F is selected from a31 or a32:
wherein a31 is a single-stranded DNA molecule having the sequence of SEQ ID NO: 11;
a32 is a single-stranded DNA molecule having the sequence of the 19th to 36th nucleotides from the 5′ end of SEQ ID NO: 11;
wherein the primer ETEC-R is selected from a34 or a35:
wherein a34 is a single-stranded DNA molecule having the sequence of SEQ ID NO: 12;
a35 is a single stranded DNA molecule having the sequence of the 20th to 40th nucleotides from the 5′ end of SEQ ID NO: 12;
wherein the primer pair VII comprises a primer IBRV-F and a primer IBRV-R;
wherein the primer IBRV-F is selected from a37 or a38:
wherein a37 is a single-stranded DNA molecule having the sequence of SEQ ID NO: 13;
a38 is a single-stranded DNA molecule having the sequence of the 19th to 41th nucleotides from the 5′ end of SEQ ID NO: 13;
wherein the primer IBRV-R is selected from a′or a41:
wherein a40 is a single stranded DNA molecule having the sequence of SEQ ID NO: 14;
a41 is a single stranded DNA molecule having the sequence of the 20th to 36th nucleotides from the 5′ end of SEQ ID NO: 14;
wherein the primer pair VIII comprises a primer PPRV-F and a primer PPRV-R;
wherein the primer PPRV-F is selected from a43 or a44:
wherein a43 is a single-stranded DNA molecule having the sequence of SEQ ID NO: 15;
a44 is a single-stranded DNA molecule having the sequence of the 19th to 44th nucleotides from the 5′ end of SEQ ID NO: 15;
wherein the primer PPRV-R is selected from the a46 or a47:
wherein a46 is a single stranded DNA molecule having the sequence of SEQ ID NO: 16;
a47 is a single stranded DNA molecule having the sequence of the 20th to 36th nucleotides from the 5′ end of SEQ ID NO: 16.

2. (canceled)

3. A kit containing a primer combination; comprising a primer combination, a buffer, a MgCl₂ solution and a DNA polymerase;
wherein the primer combination consists of one or more primer pairs selected from the group consisting of primer pair I, primer pair II, primer pair III, primer pair IV, primer pair V, primer pair VI, primer pair VII and primer pair VIII;
wherein the use of the kit comprises c1, c2 and c3:
c1 is identifying eight kinds of bovine pathogens: wherein the eight kinds of bovine pathogens comprising foot and mouth disease virus, bluetongue virus, vesicular stomatitis virus, bovine viral diarrhea virus, bovine rotavirus, enterotoxigenic E. coli, infectious bovine rhinotracheitis virus and peste des petits ruminant virus;
c2 is detecting whether the pathogen to be tested is foot and mouth disease virus, bluetongue virus, vesicular stomatitis virus, bovine viral diarrhea virus, bovine rotavirus, enterotoxigenic E. coli, infectious bovine rhinotracheitis virus or peste des petits ruminant virus;
c3 is detecting whether the sample to be tested contains foot and mouth disease virus and/or bluetongue virus and/or vesicular stomatitis virus and/or bovine viral diarrhea virus and/or bovine rotavirus and/or enterotoxin E. coli and/or bovine infectious rhinotracheitis virus and/or peste des petits ruminant virus;
wherein the primer pair I comprises a primer FMDV-F and a primer FMDV-R;
wherein the primer FMDV-F is selected from a1 or a2,
wherein a1 is a single-stranded DNA molecule having the sequence of SEQ ID NO: 1;
a2 is a single-stranded DNA molecule having the sequence of the 19th to 36th nucleotides from the 5′ end of SEQ ID NO: 1;
wherein the primer FMDV-R is selected from a4 or a5:
wherein a4 is a single stranded DNA molecule having the sequence of SEQ ID NO: 2:
a5 is a single stranded DNA molecule having the sequence of the 20th to 43th nucleotides from the 5′ end of SEQ ID NO: 2;
wherein the primer II comprises a primer BTV-F and a primer BTV-R;
wherein the primer BTV-F is selected from a7 or a8:
wherein a7 is a single stranded DNA molecule having the sequence of SEQ ID NO: 3;
a8 is a single stranded DNA molecule having the sequence of the 19th to 41th nucleotides from the 5′ end of SEQ ID NO: 3;
wherein the primer BTV-R is selected from a10 or a11:
wherein a10 is a single stranded DNA molecule having the sequence of SEQ ID NO: 4;
a11 is a single stranded DNA molecule having the sequence of the 20th to 37th nucleotides from the 5′ end of SEQ ID NO: 4;
wherein the primer pair III comprises a primer VSV-F and a primer VSV-R;
wherein the primer VSV-F is selected from a13 or a14:
wherein a13 is a single-stranded DNA molecule having the sequence of SEQ ID NO: 5;
a14 is a single-stranded DNA molecule having the sequence of the 19th to 38th nucleotides from the 5′ end of SEQ ID NO: 5;
wherein the primer VSV-R is selected from a16 or a17:
wherein a16 a single-stranded DNA molecule having the sequence of SEQ ID NO: 6;
a17 is a single stranded DNA molecule having the sequence of the 20th to 38th nucleotides from the 5′ end of SEQ ID NO: 6;
wherein the primer pair IV comprises a primer BVDV-F and a primer BVDV-R;
wherein the primer BVDV-F is selected from a19 or a20:
wherein a19 is a single-stranded DNA molecule having the sequence of SEQ ID NO: 7;
a20 is a single-stranded DNA molecule having the sequence of the 19th to 36th nucleotides from the 5′ end of SEQ ID NO: 7;
wherein the primer BVDV-R is selected from a22 or a23:
wherein a22 is a single-stranded DNA molecule having the sequence of SEQ ID NO: 8;
a23 is a single-stranded DNA molecule having the sequence of the 20th to 44th nucleotides from the 5′ end of SEQ ID NO: 8;
wherein the primer pair V is comprises a primer BRV-F and a primer BRV-R;
wherein the primer BRV-F is selected from a25 or a26:
wherein a25 is a single-stranded DNA molecule having the sequence of SEQ ID NO: 9;
a26 is a single-stranded DNA molecule having the sequence of the 19th to 40th nucleotides from the 5′ end of SEQ NO: 9;
wherein the primer BRV-R is selected from a28 or a29:
wherein a28 is a single stranded DNA molecule having the sequence of SEQ ID NO: 10;
a29 is a single stranded DNA molecule having the sequence of the 20th to 37th nucleotides from the 5′ end of SEQ ID NO: 10;
wherein the primer pair VI comprises a primer ETEC-F and a primer ETEC-R;
wherein the primer ETEC-F is selected from a3 1 or a32:
wherein a31 is a single-stranded DNA molecule having the sequence of SEQ ID NO: 11;
a32 is a single-stranded DNA molecule having the sequence of the 19th to 36th nucleotides from the 5′ end of SEQ ID NO: 11;
wherein the primer ETEC-R is selected from a34 or a35:
wherein a34 is a single-stranded DNA molecule having the sequence of SEQ ID NO: 12;
a35 is a single stranded DNA molecule having the sequence of the 20th to 40th nucleotides from the 5′ end of SEQ ID NO: 12;
wherein the primer pair VII comprises a primer IBRV-F and a primer IBRV-R;
wherein the primer IBRV-F is selected from a37 or a38:
wherein a37 is a single-stranded DNA molecule having the sequence of SEQ ID NO: 13;
a38 is a single-stranded DNA molecule having the sequence of the 19th to 41th nucleotides from the 5′ end SEQ ID NO: 13;
wherein the primer IBRV-R is selected from a40 or a41;
wherein a40 is a single stranded DNA molecule having the sequence of SEQ ID NO: 14;
a41 is a single stranded DNA molecule having the sequence of the 20th to 36th nucleotides from the 5′ end of SEQ ID NO: 14;
wherein the primer pair VIII comprises a primer PPRV-F and a primer PPRV-R;
wherein the primer PPRV-F is selected from a43 or a44:
wherein a43 is a single-stranded DNA molecule having the sequence of SEQ ID NO: 15;
a44 is a single-stranded DNA molecule having the sequence of the 19th to 44th nucleotides from the 5′ end of SEQ ID NO: 15;
wherein the primer PPRV-R is selected from the a46 or a47:
wherein a46 is a single stranded DNA molecule having the sequence of SEQ ID NO: 16;
a47 is a single stranded DNA molecule having the sequence of the 20th to 36th nucleotides from the 5′ end of SEQ ID NO: 16.

4. (canceled)

5. A method for identifying eight bovine pathogens, comprising the following steps d1 or d2:
wherein step d1 comprising conducting a genomic DNA extraction operation and/or an RNA extraction operation to obtain nucleic acids of the pathogen to be tested; subjecting a reverse transcription to the nucleic acids of the pathogen to be tested containing RNA or may containing RNA, and conducting a PCR amplification using the primer combination consisting of one or more primer pairs selected from the group consisting of primer pair I, printer pair II, primer pair III, primer pair IV, primer pair V, primer pair VI, primer pair VII and primer pair VIII with the DNA and/or cDNA obtained as templates;
if the amplification product contains 165-167 bp DNA fragments, the pathogen is or is candidate for foot and mouth disease virus, if the amplified product contains 135-137 bp DNA fragment, the pathogen to be tested is or is candidate for bluetongue virus, if the amplified product contains 278-281 bp DNA fragment, the pathogen to be tested is or is candidate for vesicular stomatitis virus, if the amplified product contains a DNA fragment of 308-310 bp, the pathogen to be tested is or is candidate for bovine viral diarrhea virus, if the amplified product contains a DNA fragment of 211-214 bp, the pathogen to be tested is or is candidate for bovine rotavirus, and if the amplified product contains a DNA fragment of 342-345 bp, the pathogen to be tested is or is candidate for peste des petits ruminants virus, if the amplified product contains a DNA fragment of 252-254 bp, the pathogen to be tested is or is candidate for enterotoxigenic E. coli., if the amplified product contained a DNA fragment of 187-189 bp, the pathogen to be tested is or is candidate for infectious bovine rhinotracheitis;
wherein step d2 comprising detecting whether the genomic DNA or cDNA of the pathogen to be detected contains the target sequence of the primer pair I, the target sequence of the primer pair II, the target sequence of the primer pair III, the target sequence of the primer pair IV, the target sequence of the primer pair V, the target sequence of the primer pair VI, the target sequence of the primer pair VII or the target sequence of the primer pair VIII, if the cDNA contains the target sequence of the primer pair I, the pathogen to be tested is or is candidate for foot-and-mouth disease virus, if the cDNA contains the target sequence of the primer pair II, the pathogen to be tested is or is candidate for bluetongue virus, and if the cDNA contains the target sequence of the primer pair III, the pathogen to be tested is or is candidate for vesicular stomatitis virus, if the cDNA contains the target sequence of the primer pair IV, the pathogen to be tested is or is candidate for bovine viral diarrhea virus, if the cDNA contains the target sequence of the primer pair V, the pathogen to be tested is or is candidate for bovine rotavirus, if the genomic DNA contains the target sequence of the primer pair VI, the pathogen to be tested is or is candidate for enterotoxigenic E. coli, if the cDNA contains the target sequence of the primer pair VII, the pathogen to be tested is or is candidate for infectious bovine rhinotracheitis virus, and if the cDNA contains the target sequence of the primer pair VIII, the pathogen to be tested is or is candidate for peste des petits ruminants virus;
wherein the eight kinds of bovine pathogens are foot-and-mouth disease virus, bluetongue virus, vesicular stomatitis virus, bovine viral diarrhea virus, bovine rotavirus, enterotoxin E. coli, infectious bovine rhinotracheitis virus and peste des petits ruminants virus
wherein the primer pair I comprises a primer FMDV-F and a primer FMDV-R;
wherein the primer FMDV-F is selected from a1 or a2,
wherein a1 is a single-stranded DNA molecule having the sequence of SEQ ID NO: 1;
a2 is a single-stranded DNA molecule having the sequence of the 19th to 36th nucleotides from the 5′ end of SEQ ID NO: 1;
wherein the primer FMDV-R is selected from a4 or a5:
wherein a4 is a single stranded DNA molecule having the sequence of SEQ ID NO: 2;
a5 is a single stranded DNA molecule having the sequence of the 20th to 43th nucleotides from the 5′ end of SEQ ID NO: 2;
wherein the primer pair III comprises a primer BTV-F and a primer BTV-R;
wherein the primer BTV-F is selected from a7 or a8:
wherein a7 is a single stranded DNA molecule having the sequence of SEQ ID NO: 3;
a8 is a single stranded DNA molecule having the sequence of the 19th to 41th nucleotides from the 5′ end of SEQ ID NO: 3;
wherein the primer BTV-R is selected from a10 or a11:
wherein a10 is a single stranded DNA molecule having the sequence of SEQ ID NO: 4;
a11 is a single stranded DNA molecule having the sequence of the 20th to 37th nucleotides from the 5′ end of SEQ ID NO: 4;
wherein the primer pair III comprises a primer VSV-F and a primer VSV-R;
wherein the primer VSV-F is selected from a13 or a14:
wherein a13 is a single-stranded DNA molecule having the sequence of SEQ ID NO: 5;
a14 is a single-stranded DNA molecule having the sequence of the 19th to 38th nucleotides from the 5′ end of SEQ ID NO: 5;
wherein the primer VSV-R is selected from a16 or a17:
wherein a16 is a single-stranded DNA molecule having the sequence of SEQ ID NO: 6;
a17 is a single stranded DNA molecule having the sequence of the 20th to 38th nucleotides from the 5′ end of SEQ ID NO: 6;
wherein the primer pair IV comprises a primer BVDV-F and a primer BVDV-R;
wherein the primer BVDV-F is selected from a19 or a20:
wherein a19 is a single-stranded DNA molecule having the sequence of SEQ ID NO: 7;
a20 is a single-stranded DNA molecule having the sequence of the 19th to 36th nucleotides from the 5′ end of SEQ ID NO: 7;
wherein the primer BVDV-R is selected from a22 or a23:
wherein a22 is a single-stranded DNA molecule having the sequence of SEQ ID NO: 8;
a23 is a single-stranded DNA molecule having the sequence of the 20th to 44th nucleotides from the 5′ end of SEQ ID NO: 8;
wherein the primer pair V is comprises a primer BRV-F and a primer BRV-R;
wherein the primer BRV-F is selected from a25 or a26:
wherein a25 is a single-stranded DNA molecule having the sequence of SEQ ID NO: 9;
a26 is a single-stranded DNA molecule having the sequence of the 19th to 40th nucleotides from the 5′ end of SEQ ID NO: 9;
wherein the primer BRV-R is selected from a28 or a29:
wherein a28 is a single stranded DNA molecule having the sequence of SEQ ID NO: 10;
a29 is a single stranded DNA molecule having the sequence of the 20th to 37th nucleotides from the 5′ end of SEQ ID NO: 10;
wherein the primer pair VI comprises a primer ETEC-F and a primer ETEC-R;
wherein the primer ETEC-F is selected from a31 or a32;
wherein a31 is a single-stranded DNA molecule having the sequence of SEQ ID NO: 11;
a32 is a single-stranded DNA molecule having the sequence of the 19th to 36th nucleotides tom the 5′ end of SEQ ID NO: 11;
wherein the primer ETEC-R is selected from a34 or a35:
wherein a34 is a single-stranded DNA molecule having the sequence of SEQ ID NO: 12;
a35 is a single stranded DNA molecule having the sequence of the 20th to 40th nucleotides from the 5′ end of SEQ ID NO: 12;
wherein the primer pair VII comprises a primer IBRV-F and a primer IBRV-R;
wherein the primer IBRV-F is selected from a37 or a38:
wherein a37 is a single-stranded DNA molecule having the sequence of SEQ ID NO: 13;
a38 is a single-stranded DNA molecule having the sequence of the 19th to 41th nucleotides from the 5′ end of SEQ ID NO: 13;
wherein the primer IBRV-R is selected from a40 or a41:
wherein a40 is a single stranded DNA molecule having the sequence of SEQ ID NO: 14;
a41 is a single stranded DNA molecule having the sequence of the 20th to 36th nucleotides from the 5′ end of SEQ ID NO: 14;
wherein the primer pair VIII comprises a primer PPRV-F and a primer PPRV-R;
wherein the primer PPRV-F is selected from a43 or a44:
wherein a43 is a single-stranded DNA molecule having the sequence of SEQ ID NO: 15;
a44 is a single-stranded DNA molecule having the sequence of the 19th to 44th nucleotides from the 5′ end of SEQ ID NO: 15;
wherein the primer PPRV-R is selected from the a46 or a47:
wherein a46 is a single stranded DNA molecule having the sequence of SEQ ID NO: 16;
a47 is a single stranded DNA molecule having the sequence of the 20th to 36th nucleotides from the 5′ end of SEQ ID NO: 16.

6. (canceled)

7. (canceled)

8. (canceled)

9. (canceled)

10. (canceled)

11. The kit according to claim 3, wherein the primers are packaged individually.
